(19) United States
(12) Patent Application Publication
(10) Pub. No.: US 2019/0367944 A1
(43) Pub. Date: Dec. 5, 2019

(54) RECOMBINANT VIRUS VECTORS FOR THE TREATMENT OF GLYCOGEN STORAGE DISEASE

(71) Applicant: The U.S.A., as represented by the Secretary, Department of Health and Human Services, Bethesda, MD (US)

(72) Inventor: Janice J. Chou, North Bethesda, MD (US)

(73) Assignee: The U.S.A., as represented by the Secretary, Department of Health and Human Services, Bethesda, MD (US)

(21) Appl. No.: 16/481,430

(22) PCT Filed: Jan. 30, 2018
(86) PCT No.: PCT/US2018/015957

§ 371 Date: Jul. 26, 2019

Related U.S. Application Data
(60) Provisional application No. 62/451,963, filed on Jan. 30, 2017.

(51) Int. Cl. C12N 15/86; C07K 14/705

(52) U.S. Cl. CPC C12N 15/86; A61K 48/00; C07K 14/705

(57) ABSTRACT

Recombinant viruses, such as adeno-associated virus (rAAV) or lentivirus, for the treatment of glycogen storage disease type Ib (GSD-Ib) are described. The recombinant viruses use either the human glucose-6-phosphatase (G6PC) promoter/enhancer (GPE) or the minimal human G6PT promoter/enhancer (miGT) to drive expression of human glucose-6-phosphate transporter (G6PT). The disclosed vectors are capable of delivering the G6PT transgene to the liver and correcting metabolic abnormalities in a murine model of GSD-Ib. The recombinant virus-treated mice maintained glucose homeostasis, tolerated a long fast, and did not elicit anti-G6PT antibodies. Methods of treating a subject diagnosed with GSD-Ib using the recombinant viruses is further described.

### CROSS REFERENCE TO RELATED APPLICATIONS

This application claims the benefit of U.S. Provisional Application No. 62/451,963, filed Jan. 30, 2017, which is herein incorporated by reference in its entirety.

### FIELD

This disclosure concerns gene therapy vectors for the treatment of glycogen storage disease, particularly glycogen storage disease type Ib.

### BACKGROUND

Glycogen storage disease type Ib (GSD-Ib, MIM232220) is caused by a deficiency in the ubiquitously expressed glucose-6-phosphate (G6P) transporter (G6PT or SLC37A4), which translocates G6P from the cytoplasm into the lumen of the endoplasmic reticulum (ER) (Chou et al., Curr Mol Med 2: 121-143, 2002; Chou et al., Net Rev Endocrinol 6: 676-688, 2010). Inside the ER, G6P is hydrolyzed to glucose and phosphate by either the liver/kidney/intestine-restricted glucose-6-phosphatase-α (G6Pase-α or G6PC) or the ubiquitously expressed G6Pase-β. G6PT and G6Pase are functionally co-dependent and form the G6PT/G6Pase complexes. The G6PT/G6Pase-α complex maintains interprandial blood glucose homeostasis. A deficiency of either protein results in an abnormal metabolic phenotype characterized by fasting hypoglycemia, hepatomegaly, nephromegaly, hyperlipidemia, hyperuricemia, lactic acidemia, and growth retardation. The G6PT/G6Pase-β complex maintains neutrophil/macrophage homeostasis and function, and a deficiency of either protein results in neutropenia and myeloid dysfunction (Chou et al., Curr Mol Med 2: 121-143, 2002; Chou et al., Nat Rev Endocrinol 6: 676-688, 2010). Therefore GSD-Ib is not only a metabolic but also an immune disorder characterized by impaired glucose homeostasis, neutropenia, and myeloid dysfunction. Untreated GSD-Ib is juvenile lethal. Strict compliance with dietary therapies (Greene et al., N Engl J Med 294: 423-425, 1976; Chen et al., N Engl J Med 310: 171-175, 1984), along with granulocyte colony stimulating factor (G-CSF) therapy (Visser et al., J Peditr 137: 187-191, 2000; Visser et al., Eur J Pediatr 161 (Suppl 1): S83-S87, 2002) have enabled GSD-Ib patients to attain near normal growth and pubertal development. However, no current therapy is able to address the long-term complication of hepatocellular adenoma (HCA) that develops in 75% of GSD-I patients over 25 years-old (Chou, et al., Curr Mol Med 2: 121-143, 2002; Chou et al., Nat Rev Endocrinol 6: 676-688, 2010; Rake et al., Eur J Pediatr 161 (Suppl 1): S20-S34, 2002; Franco et al., J Inherit Metab Dis 28: 153-162, 2005).

### SUMMARY

Disclosed herein are recombinant nucleic acid molecules, recombinant vectors, such as adeno-associated virus (AAV) vectors or lentivirus vectors, and recombinant viruses that can be used in gene therapy applications for the treatment of glycogen storage disease, specifically GSD-Ib.

Provided herein are recombinant nucleic acid molecules that include a human glucose-6-phosphate transporter (G6PT) coding sequence operably linked to either a human glucose-6-phosphatase (G6PC) promoter/enhancer (GPE) sequence, or a minimal G6PF promoter/enhancer (miGT) sequence.

Also provided are vectors that include a recombinant nucleic acid molecule disclosed herein. In some embodiments, the vector is an AAV vector. In other embodiments, the vector is a lentivirus vector. Further provided are isolated host cells comprising the recombinant nucleic acid molecules or vectors disclosed herein. For example, the isolated host cells can be cells suitable for propagation of AAV or lentivirus.

Further provided are recombinant AAV (rAAV) or recombinant lentivirus that include a recombinant nucleic acid molecule disclosed herein. Compositions that include a rAAV or a recombinant lentivirus disclosed herein and a pharmaceutically acceptable carrier are also provided.

Also provided herein are methods of treating a subject diagnosed with a glycogen storage disease. In some embodiments, the method includes selecting a subject with GSD-Ib and administering to the subject a therapeutically effective amount of a recombinant virus or composition disclosed herein.

The foregoing and other objects, features, and advantages of the disclosure will become more apparent from the following detailed description, which proceeds with reference to the accompanying figures.

### BRIEF DESCRIPTION OF THE DRAWINGS

FIGS. 1A-1E. Phenotype analysis of 6-week-old wild-type and rAAV-treated G6pt−/− mice. (FIG. 1A) Liver microsomal G6P uptake activity. The data were obtained from wild-type (+/+, n=8), GPE (n=12) and miGT (n=12) mice. (FIG. 1B) Blood glucose levels. (FIG. 1C) Body weight (BW), liver weight (LW), and LW/BW of mice. The data were obtained from wild-type (+/+, n=24), GPE (n=13) and miGT (n=15) mice. (FIG. 1D) Blood neutrophil counts expressed as percent of white blood cells. The data were obtained from wild-type (+/+, n=16), GPE (n=6) and miGT (n=7) mice. (FIG. 1E) Bone marrow neutrophil respiratory burst activity in response to 200 ng/mL of phorbol myristate acetate (PMA) and calcium flux activity in response to 10⁻⁶ M of f-Met-Leu-Phe (fMLP). The data were obtained from wild-type (+/+, n=3), GPE (n=2) and miGT (n=2) mice. Data represent the mean±SEM. *p<0.05, **p<0.005.

FIGS. 2A-2C. Biochemical analyses of 60-78 week-old wild-type and rAAV-treated G6pt−/− mice. (FIG. 2A) Liver microsomal G6P uptake activity in the rAAV-treated G6pt−/− mice is shown at the indicated ages in weeks (W). The mice were grouped based on the gene construct and viral dosages: GPE (n=6), GPE-low (n=9) and miGT (n=15) mice. Two major subgroups emerged for mice expressing 44-62% (G6PT/44-62%, n=6) and 3-22% (G6PT/3-22%, n=24) of normal hepatic G6PT activity. The G6PT/44-62% mice included GPE mice and the G6PT/3-22% mice (n=24) included GPE-low and miGT mice. Hepatic microsomal G6P uptake activity in 60-78 week-old wild-type mice (n=30) averaged 123±6 units (pmol/min/mg). (FIG. 2B) Hepatic microsomal G6P uptake activity and its relationship to vector genome copy numbers. (FIG. 2C) Hepatic G6pc mRNA expression and microsomal G6Pase-α enzymatic activity of 60-78-week-old wide-type (+/+, n=30), G6PT/44-62% (n=6), and G6PT/3-22% (n=24) mice. Data represent the mean±SEM. *p<0.05, **p<0.005.

FIGS. 3A-3E. Phenotype analysis and fasting blood glucose tolerance profiles of 60-78-week-old wild-type and rAAV-treated G6pt−/− mice. The data were analyzed from wide-type (+/+, n=30), G6PT/44-62% (n=6), and G6PT/3-22% (GPE-low, n=9 and miGT, n=15) mice. (FIG. 3A) Blood glucose, cholesterol, triglyceride, uric acid, and lactic acid levels. (FIG. 3B) BW and body fat values. (FIG. 3C) LW/BW ratios. (FIG. 3D) H&E stained liver sections and hepatic glycogen contents. Each plate represents an individual mouse; two mice are shown for each treatment. Two representative H&E stained HCA are shown in the GPE-low and the miGT mice. Scale bar=200 μm. The arrow denotes HCA. (FIG. 3E) Glucose tolerance test profiles. Data represent the mean±SEM. *p<0.05, **p<0.005.

FIGS. 4A-4F. Phenotype, glucose tolerance, insulin tolerance, and anti-G6PT antibody analysis of 60-78 week-old wild-type and rAAV-treated G6pt−/− mice. The data were analyzed from wide-type (+/+, n=30), G6PT/44-62% (n=6), and G6PT/3-22% (GPE-low, n=9 and miGT, n=15) mice. (FIG. 4A) Fasting glucose tolerance profiles and the 24 hour fasted blood glucose levels. (FIG. 4B) Hepatic glucose levels. (FIG. 4C) Hepatic lactate and triglyceride contents. (FIG. 4D) Twenty-four hour fasted blood insulin levels. (FIG. 4E) Insulin tolerance test profiles. Values are reported as a percent of respective level of each group at zero time. (FIG. 4F) Antibodies against human G6PT. Microsomal proteins from Ad-human (h) G6PT infected COS-1 cells were electrophoresed through a single 12% polyacrylamide-SDS gel and transferred onto a PVDF membrane. Membrane strips, representing individual lanes on the gel were individually incubated with the appropriate mouse serum. A polyclonal anti-human G6PT antibody that also recognizes murine G6PT was used as a positive control. Lanes 1, 2, 13, 14: anti-hG6PT antiserum; lanes 3, 5, 7, 9, 11, 15, 17, 19, 21: serum samples (1:50 dilution) from wild-type mice, or serum samples (1:50 dilution) from G6PT/44-62% (lanes 4, 6, 8), GPE-low (lanes 10, 12, 16), and miGT (lanes 18, 20, 22) mice. Data represent the mean±SEM. *p<0.05, **p<0.005.

FIGS. 5A-5E. Analysis of hepatic carbohydrate response element binding protein (ChREBP) signaling in 60-78-week-old wild-type and rAAV-treated G6pt−/− mice. For quantitative RT-PCR and hepatic G6P levels, the data represent the mean±SEM for 60-78-week-old wild-type (n=30), G6FT/44-62% (n=6), and G6FT/3-22% (GPE-low, n=9 and miGT, n=15) mice. (FIG. 5A) Hepatic G6P levels. (FIG. 5B) Quantification of ChREBP mRNA by real-time RT-PCR. (FIG. 5C) Immunohistochemical analysis of hepatic ChREBP nuclear localization and quantification of nuclear ChREBP-translocated cells. Scale bar=50 μm. The data represent the mean±SEM for wild-type (+/+, n=7), G6PT/44-62% (n=4), and G6PT/3-22% (n=15) mice. (FIG. 5D) Quantification of mRNA for Acc1, Fasn, and Scd1 by real-time RT-PCR. (FIG. 5E) Western blot analysis of ACC1, FASN, and SCD1, β-actin and quantification of protein levels by densitometry of wild-type (+/+, n=17), G6PT/44-62% (n=5), and G6PT/3-22% (n=12) mice. Data represent the mean±SEM. *p<0.05, **p<0.005.

FIGS. 6A-6B. Analysis of hepatic Akt and FGF21 in 60-78-week-old wild-type and rAAV-treated G6pt−/− mice. For quantitative RT-PCR. the data represent the mean±SEM for 60-78-week-old wild-type (n=30), G6PT/44-62% (n=6), and G6PT/3-22% (GPE-low, n=9 and miGT. n=15) mice. (FIG. 6A) Quantification of mRNA for Akt. Western blot analysis of Akt, p-Akt-S473. p-Akt-T308, and β-actin and quantification protein levels by densitometry of wild-type (+/+, n=17), G6PT/44-62% (n=5). and G6PT/3-22% (n=12) mice. (FIG. 6B) Quantification of mRNA for FGF21, Western blot analysis of FGF21. β-actin and quantification protein levels by densitometry of wild-type (+/+, n=17). G6PT/44-62% (n=5). and G6PT/3-22% (n=12) mice. Data represent the mean±SEM. *p<0.05, **p<0.005.

FIGS. 7A-7B. Analysis of hepatic sirtuin 1 (SIRT1) and AMP-activated protein kinase (AMPK) signaling. (FIG. 7A) Western blot analysis of SIRT1, p-AMPK-T172, AMPK and β-actin with quantification of protein levels by densitometry in 60-78-week-old wild-type (+/+, n=17), G6PT/44-62% (n=5) and G6PT/3-22% (n=12) mice. (FIG. 7B) Hepatic NAD+ levels in wild-type (n=17), G6PT/44-62% (n=5) and G6PT/3-22% (n=12) mice. Data represent the mean±SEM. *P<0.05, **P<0.005.

FIGS. 8A-8B. Analysis of hepatic signal transducer and activator of transcription 3 (STAT3) and nuclear factor kappa B (NFB) signaling. (FIG. 8A) Quantification of mRNA for Stat3 and Nfkb by qPCR in 60-78-week-old wild-type (+/+, n=30). G6PT/44-62% (n=6) and G6PT/3-22% (n=24) mice. (FIG. 8B) Western blot analysis of STAT3-Y705. STAT3, Ac-NFκB-p65-K310 and β-actin with quantitication of protein levels by densitometry in wild-type (+/+, n=17), G6PT/44-62% (n=5) and G6PT/3-22% (n=12) mice. Data represent the mean±SEM. *p<0.05. **p<0.005.

FIG. 9. Western blot analysis of E-cadherin, N-cadherin, Slug and β-actin with quantification of protein levels by densitometry in 60-78-week-old wild-type (+/+, n=17), G6PT/44-62% (n=5) and G6PT/3-22% (n=12) mice. Data represent the mean±SEM. *p<0.05, **p<0.005.

FIGS. 10A-10B. Analysis of hepatic β-klotho expression. (FIG. 10A) Quantification of mRNA for β-klotho by qPCR in 60-78-week-old wild-type (+/+, n=30), G6PT/44-62% (n=6) and G6PT/3-22% (n=24) mice. (FIG. 10B) Western blot analysis of and β-klotho and β-actin with quantification of protein levels by densitometry in 60-78-week-old wild-type (+/+, n=17), G6PT/44-62% (n=5) and G6PT/3-22% (n=12) mice. Data represent the mean±SEM. *p<0.05. **p<0.005.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on January 22, 18.0 KB, which is incorporated by reference herein. In the accompanying sequence listing:

SEQ ID NO: 1 is the nucleotide sequence of pTR-GPE-human G6PT having the following features:

ITR—nucleotides 17-163

G6PC promoter/enhancer (GPE)—nucleotides 182-3045

Intron—nucleotides 3185-3321

G6PT coding sequence—nucleotides 3366-4655

ITR—nucleotides 4868-5003.

SEQ ID NO: 2 is the nucleotide sequence of pTR-miGT-human G61T having the following features:

ITR—nucleotides 17-163

miGT—nucleotides 182-792

Intron—nucleotides 924-1560

G6PT coding sequence—nucleotides 1105-1938

ITR—nucleotides 2171-2316.

### DETAILED DESCRIPTION

### I. Abbreviations

AAV adeno-associated virus

AMPK AMP-activated protein kinase

BIV bovine immunodeficiency virus

BW body weight

CAEV caprine arthritis-encephalitis virus

CBA chicken β-actin

ChREBP carbohydrate response element binding protein

CMV cytomegalovirus

EIAV equine infectious anemia virus

EMT epithelial-mesenchymal transition

ER endoplasmic reticulum

FIV feline immunodeficiency virus

fMLP f-Met-Leu-Phe

G6P glucose-6-phosphate

G6PC glucose-6-phosphatase, catalytic subunit

G6PT glucose-6-phosphate transporter

GPE G6PC promoter/enhancer

GSD glycogen storage disease

H&E hematoxylin & eosin

HCA hepatocellular adenoma

HIV human immunodeficiency virus

ITR inverted terminal repeat

LW liver weight

miGT minimal G6PT promoter/enhancer

NKκB nuclear factor kappa B

ORF open reading frame

PMA phorbol myristate acetate

rAAV recombinant AAV

SEM standard error of the mean

SIRT1 sirtuin 1

SIV simian immunodeficiency virus

STAT3 signal transducer and activator of transcription 3

vp viral particles

### II. Terms and Methods

Unless otherwise noted, technical terms are used according to conventional usage. Definitions of common terms in molecular biology may be found in Benjamin Lewin, Genes V. published by Oxford University Press, 1994 (ISBN 0-19-854287-9); Kendrew et al. (eds.), The Encyclopedia of Molecular Biology, published by Black-well Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 11 recognized serotypes of AAV (AAV1-11).

Administration/Administer: To provide or give a subject an agent, such as a therapeutic agent (e.g. a recombinant AAV), by any effective route. Exemplary routes of administration include, but are not limited to, injection (such as subcutaneous, intramuscular, intradermal, intraperitoneal, intravenous, or renal vein injection), oral, intraductal, sublingual, rectal, transdermal, intranasal, vaginal and inhalation routes.

Enhancer: A nucleic acid sequence that increases the rate of transcription by increasing the activity of a promoter.

Glucose-6-phosphatase catalytic subunit (G6PC): A gene located on human chromosome 17q21 that encodes glucose-6-phosphatase-α (G6Pase-α). G6Pase-α is a 357 amino acid hydrophobic protein having 9 helices that anchor it in the endoplasmic reticulum (Chou et al., Nat Rev Endocrinol 6:676-688, 2010). The G6Pase-α protein catalyzes the hydrolysis of glucose 6-phosphate to glucose and phosphate in the terminal step of gluconeogenesis and glycogenolysis and is a key enzyme in glucose homeostasis. Deleterious mutations in the G6PC gene cause glycogen storage disease type Ia (GSD-Ia), which is a metabolic disorder characterized by severe fasting hypoglycemia associated with the accumulation of glycogen and fat in the liver and kidneys.

Glucose-6-phosphate transporter (G6PT): A gene located on human chromosome 11q23.3. The G6PT gene encodes a protein that regulates glucose-6-phosphate transport from the cytoplasm to the lumen of the ER in order to maintain glucose homeostasis. Mutations in the G6PT gene are associated with glycogen storage disease type Ib. G6PT is also known as solute carrier family 37 member 4 (SLC37A4).

Glycogen storage disease (GSD): A group of diseases that result from defects in the processing of glycogen synthesis or breakdown within muscles, liver and other tissues. GSD can either be genetic or acquired. Genetic GSD is caused by any inborn error of metabolism involved in these processes. There are currently 11 recognized glycogen storage diseases (GSD type I, II, III, IV, V, VI, VII, IX, XI, XII and XIII). GSD-I consists of two autosomal recessive disorders, GSD-Ia and GSD-Ib (Chou et al., Nat Rev Endocrinol 6:676-688, 2010). GSD-Ia results from a deficiency in glucose-6-phosphatase-α. Deficiencies in the glucose-6-phosphate transporter (G6PT) are responsible for GSD-Ib.

Glycogen storage disease type Ib (GSD-Ib): An autosomal recessive disorder caused by deficiencies in glucose-6-phosphate transporter (G6PT), a ubiquitously expressed endoplasmic reticulum (ER) protein that translocate G6P from the cytoplasm into the ER lumen. GSD-Ib is both a metabolic disorder and an immune disorder. GSD-Ib metabolic abnormalities include fasting hypoglycemia, hepatomegaly, nephromegaly, hyperlipidemia, hyperuricemia, lactic acidemia and growth retardation. Although dietary therapies for GSD-Ib that significantly alleviate the metabolic abnormalities of GSD-Ib are available, patients continue to suffer from long-term complications of GSD-Ib, such as hepatocellular adenoma/carcinoma and renal disease. The GSD-Ib immunological abnormalities include neutropenia and myeloid dysfunction. Neutrophils from GSD-Ib patients exhibit impairment of chemotaxis, calcium mobilization, respiratory burst, and phagocytotic activities. As a result, recurrent bacterial infections are commonly seen and up to 77% of patients manifesting neutropenia also develop inflammatory bowel disease (IBD), indistinguishable from idiopathic Crohn's disease (Visser et al., J Pediatr 137:187-191, 2000; Dieckgraefe et al., Eur J Pediatr 161:S88-S92, 2002). As used herein, “treating GSD-Ib” refers to a therapeutic intervention that ameliorates one or more signs or symptoms of GSD-Ib or a pathological condition associated with GSD-Ib. Thus, “treating GSD-Ib” can include treating any metabolic or immune dysfunction associated with GSD-Ib, such as, but not limited to, hypoglycemia, hepatomegaly, nephromegaly, hyperlipidemia, hyperuricemia. lactic academia, growth retardation, neutropenia. myeloid dysfunction and IBD.

Intron: A stretch of DNA within a gene that does not contain coding information for a protein. Introns are removed before translation of a messenger RNA.

Inverted terminal repeat (ITR): Symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV integrating vectors.

Isolated: An “isolated” biological component (such as a nucleic acid molecule, protein, virus or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Lentivirus: A genus of retroviruses characterized by a long incubation period and the ability to infect non-dividing cells. Lentiviruses are attractive gene therapy vectors due to their ability to provide long-term, stable gene expression and infect non-dividing cells. Examples of lentiviruses include human immunodeficiency virus (HIV), simian immunodeficiency virus (SIV). feline immunodeficiency virus (FIV), bovine immunodeficiency virus (BIV), caprine arthritis-encephalitis virus (CAEV) and equine infectious anemia virus (EIAV).

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carrier: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill. tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease (such as GSD-Ib) refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Promoter: A region of DNA that directs/initiates transcription of a nucleic acid (e.g. a gene). A promoter includes necessary nucleic acid sequences near the start site of transcription. Typically, promoters are located near the genes they transcribe. A promoter also optionally includes distal enhancer or repressor elements which can be located as much as several thousand base pairs from the start site of transcription.

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

Similarly, a recombinant virus is a virus comprising sequence (such as genomic sequence) that is non-naturally occurring or made by artificial combination of at least two sequences of different origin. The term “recombinant” also includes nucleic acids, proteins and viruses that have been altered solely by addition, substitution, or deletion of a portion of a natural nucleic acid molecule, protein or virus. As used herein. “recombinant AAV” refers to an AAV particle in which a recombinant nucleic acid molecule (such as a recombinant nucleic acid molecule encoding G6PT) has been packaged.

Sequence identity: The identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene. 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mo. Bio. 215:403-10, 1990. presents a detailed consideration of sequence alignment methods and homology calculations.

The NCBI Basic Local Alignment Search Tool (BLAST) (Altschul et al., J. Mot. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

Serotype: A group of closely related microorganisms (such as viruses) distinguished by a characteristic set of antigens.

Subject: Living multi-cellular vertebrate organisms, a category that includes human and non-human mammals.

Synthetic: Produced by artificial means in a laboratory, for example a synthetic nucleic acid can be chemically synthesized in a laboratory.

Therapeutically effective amount: A quantity of a specified pharmaceutical or therapeutic agent (e.g. a recombinant AAV) sufficient to achieve a desired effect in a subject, or in a cell, being treated with the agent. The effective amount of the agent will be dependent on several factors, including, but not limited to the subject or cells being treated, and the manner of administration of the therapeutic composition.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is a lentivirus vector or an AAV vector.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications. patents, and other references mentioned herein are incorporated by reference in their entirety. In case of conflict, the present specification, including explanations of terms, will control. In addition, the materials, methods, and examples are illustrative only and not intended to be limiting.

### III. Introduction

GSD-Ib (G6pt−/−) mice manifest both the metabolic and myeloid dysfunctions characteristic of human GSD-Ib (Chen et al., Hum Mol Genet 12: 2547-2558, 2003). When left untreated, the G6pt−/− mice rarely survive weaning, reflecting the juvenile lethality seen in human patients. Previous studies have shown that systemic administration of a pseudotyped AAV2/8 vector expressing human G6PT directed by the chicken β-actin (CBA) promoter/CMV enhancer, delivers the G6PT transgene primarily to the liver. In doing so, it normalizes metabolic abnormalities in murine GSD-Ib. However. of the five treated G6pt−/− mice that survived for 51-72 weeks, two (40%) developed multiple HCAs with one undergoing malignant transformation Yiu et al., J Hepatol 51: 909-917, 2009.

Studies have shown that the choice of transgene promoter can impact targeting efficiency, tissue-specific expression, and the level of immune response or tolerance to the therapy (Ziegler et al., Mol Ther 15: 492-500, 2007; Franco et al., Mol Ther 12: 876-884, 2005). Indeed, for the related disease GSD-Ia, caused by a deficiency in G6Pase-α enzyme activity, a G6Pase-α-expressing rAAV vector directed by the native 2.8-kb human G6PC promoter/enhancer (GPE) provides sustained correction of metabolic abnormalities in murine GSD-Ia with no evidence of HCA (Lee et al., Hepatology 56: 1719-1729, 2012; Kim et al., Hum Mol Genet 24: 5115-5125, 2015). Moreover, the gluconeogenic tissue-specific GPE does not elicit the humoral response that was observed for the CBA promoter/CMV enhancer (Yiu et al., Mol Ther 18:1076-1084, 2010).

The vectors disclosed herein use either the GPE or the minimal G6PT promoter/enhancer (miGT) consisting of nucleotides −610 to −1 upstream of the +1 nucleotide of the G6PT coding sequence (Hiraiwa and Chou, DNA Cell Biol 20: 447-453, 2001). The studies described herein examined the safety and efficacy of liver-directed gene therapy in G6pt−/− mice using rAAV-GPE-G6PT and rAAV-miGT-G6PT, which are rAAV8 vectors directed by the human G6PC and G6PT promoter/enhancer, respectively. The threshold of hepatic G6PT activity required to prevent tumor formation was also examined. In a 60-78 week-study, it was shown that while both vectors delivered the G6PT transgene to the liver and corrected metabolic abnormalities in murine GSD-Ib, the rAAV-GPE-G6PT vector had greater efficacy. Using dose titration to control the level of G6PT activity restored, it was shown that rAAV-treated G6pt−/− mice expressing 3-62% of normal hepatic G6PT activity maintained glucose homeostasis, tolerated a long fast, and did not elicit anti-G6PT antibodies. However, G6pt−/− mice with <6% of normal hepatic G6PT activity restored were at risk of developing hepatic tumors. It is also shown herein that restoration of hepatic G6PT expression up to 62% of wild type activity conferred protection against developing age-related obesity and insulin resistance that is found in wild-type mice.

### IV. Overview of Several Embodiments

Described herein are recombinant nucleic acid molecules, recombinant vectors, such as AAV and lentivirus vectors, and recombinant viruses, such as recombinant AAV and recombinant lentivirus, that can be used in gene therapy applications for the treatment of glycogen storage disease, specifically GSD-Ib.

Provided herein are recombinant nucleic acid molecules that include a human glucose-6-phosphate transporter (G6PT) coding sequence operably linked to a human glucose-6-phosphatase (G6PC) promoter/enhancer (GPE) sequence. In some embodiments, the human G6PT coding sequence is at least 80%, at least 85%, at least 90%, at least 95%, at least 96%, at least 97%, at least 98% or at least 99% identical to nucleotides 3366-4655 of SEQ ID NO: 1. In some examples, the human G6PT coding sequence comprises or consists of nucleotides 3366-4655 of SEQ ID NO: 1. In some embodiments, the GPE sequence is at least 80%, at least 85%, at least 90%. at least 95%, at least 96%, at least 97%, at least 98% or at least 99% identical to nucleotides 182-3045 of SEQ ID NO: 1. In some examples, the GPE sequence comprises or consists of nucleotides 182-3045 of SEQ ID NO: 1. In particular examples, the recombinant nucleic acid molecule is at least 80%, at least 85%, at least 90%, at least 95%, at least 96%, at least 97%, at least 98% or at least 99% identical to nucleotides 182-4655 of SEQ ID NO: 1 or nucleotides 17-5003 of SEQ ID NO: 1. In specific examples, the recombinant nucleic acid molecule comprises or consists of nucleotides 182-4655 of SEQ ID NO: 1 or nucleotides 17-5003 of SEQ ID NO: 1. In other particular examples, the recombinant nucleic acid molecule is at least 80%, at least 85%, at least 90%, at least 95%, at least 96%, at least 97%, at least 98% or at least 99% identical to SEQ ID NO: 1. In specific non-limiting examples, the recombinant nucleic acid molecule comprises or consists of SEQ ID NO: 1.

Also provided herein are recombinant nucleic acid molecules that include a human G6PT coding sequence operably linked to a minimal G6PT promoter/enhancer (miGT) sequence. In some embodiments, the human G6PT coding sequence is at least 80%, at least 85%, at least 90%, at least 95%, at least 96%, at least 97%, at least 98% or at least 99% identical to nucleotides 1105-1938 of SEQ ID NO: 2. In some examples, the human G6PT coding sequence comprises or consists of nucleotides 1105-1938 of SEQ ID NO: 2. In some embodiments, the miGT sequence is at least 80%, at least 85%. at least 90%, at least 95%, at least 96%, at least 97%, at least 98% or at least 99% identical to nucleotides 182-792 of SEQ ID NO: 2. In some examples, the miGT sequence comprises or consists of nucleotides 182-792 of SEQ ID NO: 2. In particular examples, the recombinant nucleic acid molecule is at least 80%, at least 85%, at least 90%, at least 95%, at least 96%, at least 97%, at least 98% or at least 99% identical to nucleotides 182-1938 of SEQ ID NO: 2 or nucleotides 17-2316 of SEQ ID NO: 2. In specific examples, the recombinant nucleic acid molecule comprises or consists of nucleotides 182-1938 of SEQ ID NO: 2 or nucleotides 17-2316 of SEQ ID NO: 2. In other particular examples, the recombinant nucleic acid molecule is at least 80%, at least 85%, at least 90%, at least 95%, at least 96%, at least 97%, at least 98% or at least 99% identical to SEQ ID NO: 2. In specific non-limiting examples, the recombinant nucleic acid molecule comprises or consists of SEQ ID NO: 2.

Further provided are vectors comprising the recombinant nucleic acid molecules disclosed herein. In some embodiments, the vector is an AAV vector. The AAV serotype can be any suitable serotype for delivery of transgenes to a subject. In some examples, the AAV vector is a serotype 8 AAV (AAV8). In other examples the AAV vector is a serotype 1, 2, 3, 4, 5, 6, 7, 9, 10, 11 or 12 vector (i.e. AAV1, AAV2, AAV3, AAV4, AAV5, AAV6, AAV7, AAV9, AAV10, AAV11 or AAV12). In yet other examples, the AAV vector is a hybrid of two or more AAV serotypes (such as, but not limited to AAV2/1, AAV2/7, AAV2/8 or AAV2/9). The selection of AAV serotype will depend in part on the cell type(s) that are targeted for gene therapy. For treatment of GSD-Ib, the liver and kidney are the primary target organs. In other embodiments, the vector is a lentivirus vector. In some examples, the lentivirus vectors is an HIV, SIV, FIV, BIV, CAEV or EIAV vector.

Also provided herein are isolated host cells comprising the recombinant nucleic acid molecules or vectors disclosed herein. For example, the isolated host cell can be a cell (or cell line) appropriate for production of recombinant AAV (rAAV) or recombinant lentivirus. In some examples, the host cell is a mammalian cell, such as a HEK-293, HEK293T, BHK, Vero, RD, HT-1080, A549, COS-1, Cos-7, ARPE-19, or MRC-5 cell.

Further provided are rAAV comprising a recombinant nucleic acid molecule disclosed herein. In some embodiments, the rAAV is rAAV8 and/or rAAV2. However, the AAV serotype can be any other suitable AAV serotype, such as AAV1, AAV2, AAV3, AAV4, AAV5. AAV6, AAV7, AAV9, AAV10, AAV11 or AAV12, or a hybrid of two or more AAV serotypes (such as, but not limited to AAV2/1, AAV2/7, AAV2/8 or AAV2/9). Compositions comprising a rAAV disclosed herein and a pharmaceutically acceptable carrier are also provided by the present disclosure. In some embodiments. the compositions are formulated for intravenous or intramuscular administration. Suitable pharmaceutical formulations for administration of rAAV can be found, for example, in U.S. Patent Application Publication No. 2012/0219528. which is herein incorporated by reference.

Also provided are recombinant lentiviruses comprising a recombinant nucleic acid molecule disclosed herein. In some embodiments, the lentivirus is HIV, SIV, FIV, BIV, CAEV or EIAV. In particular examples, the lentivirus is HIV-1. Compositions comprising a recombinant lentivirus disclosed herein and a pharmaceutically acceptable carrier are also provided by the present disclosure. In some embodiments, the compositions are formulated for intravenous or intramuscular administration. In other embodiments, the recombinant lentivirus is formulated for ex vivo administration, such as for ex vivo administration to bone marrow cells.

Further provided are methods of treating a subject diagnosed with a glycogen storage disease, comprising selecting a subject with GSD-Ib and administering to the subject a therapeutically effective amount of a rAAV or recombinant lentivirus (or a composition comprising a rAAV or recombinant lentivirus) disclosed herein. In some embodiments, the rAAV or recombinant lentivirus is administered intravenously. In other embodiments, the recombinant virus is administered by retrograde renal vein injection (see, for example, Rocca et al., Gene Ther 21:618-628, 2014).

In some embodiments, the subject to be treated exhibits one or more metabolic abnormalities associated with GSD-Ib. In some examples, the subject suffers from fasting hypoglycemia, hepatomegaly, nephmmegaly, hyperlipidemia. hyperuricemia, lactic acidemia, and/or growth retardation. In some embodiments, the subject to be treated exhibits one or more immunological abnormalities associated with GSD-Ib. In some examples, the subject exhibits neutropenia, myeloid dysfunction, recurrent bacterial infection and/or inflammatory bowel disease (IBD).

In some embodiments, the rAAV is administered at a dose of about 1×10¹¹ to about 1×10¹⁴ viral particles (vp)/kg. In some examples, the rAAV is administered at a dose of about 1×10¹² to about 1×10¹⁴ vp/kg. In other examples, the rAAV is administered at a dose of about 5×10¹² to about 5×10¹³ vp/kg. In specific non-limiting examples, the rAAV is administered at a dose of at least about 1×10¹¹, at least about 5×10¹¹, at least about 1×10¹², at least about 5×10¹², at least about 1×10¹³, at least about 5×10¹³, or at least about 1×10¹⁴ vp/kg. In other non-limiting examples, the rAAV is administered at a dose of no more than about 5×10¹¹, no more than about 1×10¹². no more than about 5×10¹², no more than about 1×10¹³, no more than about 5×10¹³, or no more than about 1×10¹⁴ vp/kg. In specific non-limiting example, the rAAV is administered at a dose of about 0.7×10¹³ vp/kg, 2×10¹³ vp/kg, 1.4×10¹³ vp/kg or 4×10¹³ vp/kg. The rAAV can be administered in a single dose. or in multiple doses (such as 2, 3, 4, 5, 6, 7, 8, 9 or 10 doses) as needed for the desired therapeutic results.

Also provided herein is a method of treating immunological abnormalities, such as myeloid dysfunction, in a subject diagnosed with GSD-Ib. In some embodiments, the method includes obtaining bone marrow cells from the subject, transducing the bone marrow cells ex vivo with a recombinant virus disclosed herein, and infusing the transduced bone marrow cells into the subject. In some examples, the recombinant virus is a recombinant lentivirus.

### V. Recombinant AAV for Gene Therapy Applications

AAV belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency.

The AAV genome is characterized by two inverted terminal repeats (ITRs) that flank two open reading frames (ORFs). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Daya and Berns, Clin Microbiol Rev 21(4):583-593, 2008).

The left ORF of AAV contains the Rep gene, which encodes four proteins—Rep78, Rep 68, Rep52 and Rep40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio (Daya and Berns, Clin Microbiol Rev 21(4):583-593, 2008).

AAV is currently one of the most frequently used viruses for gene therapy. Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell. Because of the advantageous features of AAV, the present disclosure contemplates the use of AAV for the recombinant nucleic acid molecules and methods disclosed herein.

AAV possesses several desirable features for a gene therapy vector, including the ability to bind and enter target cells, enter the nucleus, the ability to be expressed in the nucleus for a prolonged period of time, and low toxicity. However, the small size of the AAV genome limits the size of heterologous DNA that can be incorporated. To minimize this problem, AAV vectors have been constructed that do not encode Rep and the integration efficiency element (IEE). The ITRs are retained as they are cis signals required for packaging (Daya and Berns. Clin Microbiol Rev 21(4):583-593, 2008).

Methods for producing rAAV suitable for gene therapy are well known in the art (see, for example, U.S. Patent Application Nos. 2012/0100606; 2012/0135515; 2011/0229971; and 2013/0072548; and Ghosh et al., Gene Ther 13(4):321-329, 2006), and can be utilized with the recombinant nucleic acid molecules and methods disclosed herein.

In some embodiments, the rAAV is provided as a lyophilized preparation and diluted in a virion-stabilizing composition (see, e.g., US 2012/0219528, incorporated herein by reference) for immediate or future use. Alternatively, the rAAV is provided immediately after production.

In some embodiments, the rAAV compositions contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally. auxiliary substances, such as wetting or emulsifying agents, pH buffering substances, and the like, may be present in such vehicles. Generally, excipients confer a protective effect on rAAV virions to minimize loss of rAAV, such as from formulation procedures, packaging, storage and transport. Excipients that are used to protect rAAV particles from degradative conditions include, but are not limited to, detergents, proteins. e.g., ovalbumin and bovine serum albumin, amino acids, e.g., glycine, polyhydric and dihydric alcohols, such as but not limited to polyethylene glycols (PEG) of varying molecular weights, such as PEG-200, PEG-400. PEG-600, PEG-1000, PEG-1450, PEG-3350. PEG-6000, PEG-8000 and any molecular weights in between these values, propylene glycols (PG), sugar alcohols, such as a carbohydrate, for example sorbitol. The detergent, when present, can be an anionic, a cationic, a zwitterionic or a nonionic detergent. In some embodiments, the detergent is a nonionic detergent. In some examples, the nonionic detergent is a sorbitan ester, for example, polyoxyethylenesorbitan monolaurate (TWEEN-20) polyoxyethylenesorbitan monopalmitate (TWEEN-40), polyoxyethylenesorbitan monostearate (TWEEN-60), polyoxyethylenesorbitan tristearate (TWEEN-65), polyoxyethylenesorbitan monooleate (TWEEN-80), polyoxyethylenesorbitan trioleate (TWEEN-85). In specific examples, the detergent is TWEEN-20 and/or TWEEN-80.

### VI. Lentiviral Vectors for Gene Therapy Applications

Lentiviruses are a genus of retroviruses characterized by a long incubation period and the ability to infect non-dividing cells. Lentiviruses are complex retroviruses, which, in addition to the common retroviral genes gag, pol, and env, contain other genes with regulatory or structural function. The higher complexity enables the virus to modulate its life cycle, as in the course of latent infection. Examples of lentiviruses include HIV, SIV, FIV, SIV, BIV, CAEV and EIAV.

Lentiviral vectors have been generated by multiply attenuating the HIV virulence genes, for example, the genes env, vif, vpr, vpu and nef have been deleted to make lentiviral vectors safe as gene therapy vectors for human use. Lentiviral vectors provide several advantages for gene therapy. They integrate stably into chromosomes of target cells, which is required for long-term expression, and they do not transfer viral genes, therefore avoiding the problem of generating transduced cells that can be destroyed by cytotoxic T lymphocytes. In addition, lentiviral vectors have a relatively large cloning capacity, sufficient for most envisioned clinical applications. Furthermore, lentiviruses (in contrast to other retroviruses) are capable of transducing non-dividing cells. This is very important in the context of gene therapy for some tissue types, particularly hematopoietic cells, brain, liver, lungs and muscle. For example, vectors derived from HIV-1 allow efficient in vivo and ex vivo delivery, integration and stable expression of transgenes into cells such a neurons, hepatocytes, and myocytes (Blomer et al., J Virol 71:6641-6649, 1997; Kafri et al., Nat Genet 17:314-317, 1997; Naldini et al., Science 272:263-267, 1996; Naldini et al., Curr Opin Biotechnol 9:457-463, 1998).

The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′LTR's serve to promote transcription and polyadenylation of the virion RNA's. The LTR contains all other cis-acting sequences necessary for viral replication. Lentiviruses also have additional genes, including vif, vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

A number of different lentiviral vectors, packaging cell lines and methods of generating lentiviral gene therapy vectors are known in the art (see, e.g., Escors and Breckpot, Arch Immunol Ther Erp 58(2):107-119, 2010; Naldini et al., Science 272:263-267, 1996; Naldini et al., Proc Natl Acad Sci USA 93:11382-11388, 1996; Naldini et al., Curr Opin Biotechnol 9:457-463, 1998; Zufferey et al., Nat Biotechnol, 15:871-875.1997; Dull et al., J Virol 72: 8463-8471, 1998; Ramezani et al., Mol Ther 2:458-469, 2000; and U.S. Pat. Nos. 5,994,136; 6,013,516; 6,165,782; 6,207,455; 6,218,181; 6,218,186; 6,277,633; 7,901,671; 8,551,773; 8,709,799; and 8,748,169, which are herein incorporated by reference). Thus, one of skill in the art is capable of selecting an appropriate lentiviral vector for the recombinant nucleic acid molecules disclosed herein.

Also provided herein are isolated cells comprising the nucleic acid molecules or vectors disclosed herein. For example, the isolated cell can be a cell (or cell line) appropriate for production of lentiviral gene therapy vectors, such as a packaging cell line. Exemplary cell lines include HeLa cells, 293 cells and PERC.6 cells.

In some embodiments, the recombinant lentivirus compositions contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally. auxiliary substances, such as wetting or emulsifying agents, pH buffering substances, and the like, may be present in such vehicles. Generally, excipients confer a protective effect on virions to minimize loss of recombinant virus, such as from formulation procedures, packaging, storage and transport. Excipients that are used to protect virus particles from degradative conditions include, but are not limited to, detergents, proteins. e.g., ovalbumin and bovine serum albumin, amino acids, e.g., glycine, polyhydric and dihydric alcohols. such as but not limited to polyethylene glycols (PEG) of varying molecular weights, such as PEG-200, PEG-400, PEG-600, PEG-1000, PEG-1450, PEG-3350, PEG-6000, PEG-8000 and any molecular weights in between these values, propylene glycols (PG), sugar alcohols, such as a carbohydrate, for example sorbitol. The detergent, when present, can be an anionic, a cationic, a zwitterionic or a nonionic detergent. In some embodiments, the detergent is a nonionic detergent. In some examples, the nonionic detergent is a sorbitan ester, for example, polyoxyethylenesorbitan monolaurate (TWEEN-20) polyoxyethylenesorbitan monopalmitate (TWEEN-40), polyoxyethylenesorbitan monostearate (TWEEN-60), polyoxyethylenesorbitan tristearate (TWEEN-65), polyoxyethylenesorbitan monooleate (TWEEN-80), polyoxyethylenesorbitan trioleate (TWEEN-85). In specific examples, the detergent is TWEEN-20 and/or TWEEN-80.

The following examples are provided to illustrate certain particular features and/or embodiments. These examples should not be construed to limit the disclosure to the particular features or embodiments described.

### EXAMPLES

### Example 1: Material and Methods

This example describes the materials and experimental procedures for the studies described in Example 2.

Construction of rAAV Vectors and Infusion of G6pt−/− Mice

The pTR-GPE-G6PT plasmid, containing human G6PT under the control of the 2.8-kb human G6PC promoter/enhancer was constructed by replacing human G6PC at 5′-SbfI and 3′ NotI sites in pTR-GPE-G6PC (Yiu et al., Mol Ther 18:1076-1084, 2010) with the human G6PT cDNA at 5′-NsiI and 3′ NotI sites. The pTR-miGT-G6PT plasmid, containing human G6PT under the control of the human G6PT minimal promoted/enhancer was constructed by replacing GPE at 5′-KpnI and 3′ HindIII sites in pTR-GPE-G6PT with the miGT at 5′-KpnI and 3′ HindIII sites. Both plasmids were verified by DNA sequencing. The rAAV-GPE-G6PT and rAAV-miGT-G6PT vectors were produced from pTR-GPE-G6PC and pTR-miGT-G6PT, respectively. For gene therapy, each vector was administered to the G6pt−/− mice in two doses—neonatally via the temporal vein and at age 4 weeks via the retro-orbital sinus. Age-matched G6pt^{+/+}/G6pt^{+/-} mice with indistinguishable phenotype were used as controls (referred collectively as wild-type or control mice).

### Microsomal G6P Uptake and Phosphohydrolase Assays

Microsomal preparations, G6P uptake and phosphohydrolase measurements were performed as described previously (Chen et al., Hum Mol Genet 12: 2547-2558, 2003; Lei et al., Net Genet 13: 203-209, 1996). In G6P uptake assays, microsomes isolated from liver were incubated for 3 minutes at 30° C. in a reaction mixture (100 μl) containing 50 mM sodium cacodylate buffer, pH 6.5, 250 mM sucrose, and 0.2 mM [U-¹⁴C]G6P (50 μCi/μmol, American Radiolabeled Chemicals, St Louis, Mo.). The reaction was stopped by filtering through a nitrocellulose membrane (Millipore, Billerica, Mass.). Microsomes permeabilized with 0.2% deoxycholate, to abolish G6P uptake, were used as negative controls. One unit of G6PT activity represents the uptake of one pmol G6P per minute per mg microsomal protein.

In phosphohydrolase assays, reaction mixtures (50 μl) containing 50 mM sodium cacodylate buffer, pH 6.5, 2 mM EDTA, 10 mM G6P, and appropriate amounts of microsomal preparations were incubated at 30° C. for 10 minutes. Disrupted microsomal membranes were prepared by incubating intact membranes in 0.2% deoxycholate for 20 minutes at 4° C. Non-specific phosphatase activity was estimated by pre-incubating disrupted microsomal preparations at pH 5 for 10 minutes at 37° C. to inactivate the acid labile G6Pase-α.

### Flow Cytometry and Functional Analysis of Bone Marrow Neutrophils

Heparinized mouse peripheral blood cells were erythrocyte-depleted and fixed in Lysis/Fix buffer (BD Biosciences. San Jose, Calif.). The resulting leukocytes were stained with a FITC-conjugated mouse monoclonal Gr-1 antibody (eBiosciences, San Diego, Calif.) and a PE-conjugated CD11b antibody (eBiosciences), and analyzed by flow cytometry using a Guava EasyCyte Mini System (Millipore).

Bone marrow cells were isolated from the femurs and tibiae of 6-week-old wild-type and rAAV-treated G6pt−/− mice, and neutrophils were purified from the bone marrow cells using the MACS separation columns system (Miltenyi Biotec, San Diego, Calif.) with Gr-1 MicroBead Kit (Miltenyi Biotec). The respiratory burst of bone marrow neutrophils was monitored by luminal-amplified chemiluminescence using the LUMIMAX™ Superoxide Anion Detection kit (Agilent Technologies, Santa Clara, Calif.) and Victor Light 1420 Luminescence counter (PerkinElmer Life & Analytical Sciences, American Fork, Utah) as described previously (Jun et al., Blood 116: 2783-2792, 2010). Neutrophils in LUMIMAX™ SOA assay medium were activated with 200 ng/ml of phorbol myristate acetate (PMA) (Sigma-Aldrich, St. Louis, Mo.). The calcium flux of bone marrow neutrophils in response to 10⁻⁶ M f-Met-Leu-Phe (fMLP) (Sigma-Aldrich) was measured using the FLIPER calcium 3 assay kit component A (Molecular Devices. Sunnyvale, Calif.) and analyzed in a Flexstation II Fluorimeter (Molecular Devices) set at 37° C. as described previously (Jun et al., Blood 116: 2783-2792, 2010).

### Phenotype Analysis

Body composition was assessed using the Bruker minispec NMR analyzer (Karlsruhe, Germany). The presence of HCA nodules in mice was confirmed by histological analysis of liver biopsy samples, using five or more separate sections per liver. Blood levels of glucose, cholesterol. triglyceride, lactate, and urate along with hepatic levels of glucose, triglyceride, lactate, and G6P were determined as described previously (Lee et al., Hepatology 56: 1719-1729, 2012; Kim et al., Hum Mol Genet 24: 5115-5125, 2015).

Glucose tolerance testing of mice consisted of fasting for 6 hours, prior to blood sampling, followed by intraperitoneal injection of a glucose solution at 2 mg/g body weight, and repeated blood sampling via the tail vein for 2 hours (Lee et al., Hepatology 56: 1719-1729, 2012). Insulin tolerance testing of mice consisted of a 4-hour fast, prior to blood sampling, followed by intraperitoneal injection of insulin at 0.25 IU/kg, and repeated blood sampling via the tail vein for 1 hour (Kim et al., Hum Mol Genet 24: 5115-5125, 2015).

### Quantitative Real-Time RT-PCR and Western-Blot Analysis

The mRNA expression was quantified by real-time RT-PCR in an Applied Biosystems 7300 Real-Time PCR System using Applied Biosystems TaqMan probes (Foster City, Calif.). Data were normalized to Rpl19 RNA. Western-blot images were detected using the LI-COR Odyssey scanner and the Image studio 3.1 software (Li-Cor Biosciences, Lincoln, Nebr.). Mouse monoclonal antibody used was: β-actin (Santa Cruz Biotechnology, Dallas, Tex.). Rabbit monoclonal antibodies used were: p-Akt-S473 and p-Akt-T308 (Cell Signaling, Danvers, Mass.); and FGF21 (Abcam, Cambridge, Mass.). Rabbit polyclonal antibodies used were: ChREBP (Novus biologicals, Littleton, Colo.); Akt, ACC and SCD-1 (Cell Signaling); and FASN (Abcam). Protein expression was quantified by densitometry using the ImageJ 1.51a software (NIH, Bethesda. Md.).

### Analysis of ChREBP Nuclear Localization

The nuclear location of ChREBP in mouse liver sections was performed as described previously (Kim et al., Hum Mol Genet 24: 5115-5125, 2015). Mouse liver paraffin sections (10 μm thickness) were treated with 0.3% hydrogen peroxide in methanol to quench endogenous peroxidases, then blocked with the Avidin/Biotin Blocking Kit (Vector Laboratories. Burlingame, Calif.). For ChREBP detection, liver sections were incubated serially with a rabbit antibody against ChREBP and a biotinylated anti-rabbit IgG (Vector Laboratories). The resulting complexes were detected with an ABC kit using the DAB Substrate (Vector Laboratories). Sections were counterstained with hematoxylin (Sigma-Aldrich) and visualized using a Zeiss Axioskop2 plus microscope equipped with 40×/0.50NA objectives (Carl Zeiss Microlmaging, Jena, Germany). Images were acquired using a Nikon DS-Fil digital camera and NIS-Elements F3.0 imaging software (Nikon, Tokyo, Japan). The percentage of cells in 10 randomly selected fields containing ChREBP positive nuclei was recorded.

### Statistical Analysis

The unpaired t-test was performed using the GraphPad Prism Program. version 4 (GraphPad Software, San Diego, Calif.). Values were considered statistically significant at p<0.05.

### Example 2: Liver-Directed Gene Therapy for Glycogen Storage Disease Type 1b

This example describes studies to examine the efficacy of G6PT gene therapy in G6pt−/− mice using recombinant adeno-associated virus (rAAV) vectors, directed by either the G6PC or the G6PT promoter/enhancer. Both vectors corrected hepatic G6PT deficiency in murine GSD-Ib, but the G6PC promoter/enhancer was more efficacious. Over a 78-week study, using dose titration of the rAAV constructs. G6pt−/− mice expressing 3-62% of normal hepatic G6PT activity exhibited a normalized liver phenotype. Two of the 12 mice expressing <6% of normal hepatic G6PT activity developed HCA. All treated mice were leaner and more sensitive to insulin than wild-type mice. Mice expressing 3-22% of normal hepatic G6PT activity exhibited higher insulin sensitivity than mice expressing 44-62%. The levels of insulin sensitivity correlated with the magnitudes of hepatic carbohydrate response element binding protein signaling activation. These studies established the threshold of hepatic G6PT activity required to prevent tumor formation and showed that mice expressing 3-62% of normal hepatic G6PT activity maintained glucose homeostasis and were protected against age-related obesity and insulin resistance.

rAAV Infusion Delivers the G6PT Transgene to the Liver

GSD-Ib mice suffer from frequent hypoglycemic seizures and despite glucose therapy to control hypoglycemia, less than 10% mice survive past weaning (Chen et al., Hum Mol Genet 12: 2547-2558, 2003). For gene therapy, each vector was administered to G6pt−/− mice in two doses, one neonatal and one at age 4 weeks, to both provide early therapy and to allow for the developmental increase in liver mass. Initially. two G6PT-expressing vectors were examined: rAAV-GPE-G6PT, a single-stranded vector directed by the 2.8-kb G6PC promoter/enhancer (Yiu et al., Mot Ther 18:1076-1084, 2010; Lee et al., Mot Genet Metab 110: 275-280, 2013) and rAAV-GT-G6PT, a single-stranded G6PT-expressing vector directed by the analogous 1.62 kb G6PT promoter/enhancer. In contrast to the efficacy observed with rAAV-GPE-G6PT (as described below), the rAAV-GT-G6PT infusion failed to sustain the survival of G6pt−/− mice, and only 4 of the 40 infused G6pt−/− mice survived to age 12 weeks. Following further promoter analysis, a different G6PT-expressing vector was constructed that includes an alternative G6PT promoter, rAAV-miGT-G6PT directed by the 610-bp G6PT promoter/enhancer, yielding a double-stranded vector to ensure proper packaging of the AAV virus. It was also anticipated that this vector construct would also benefit from an increased transduction efficiency (McCarty, Mol Ther 16: 1648-1656, 2008), which arises from bypassing the rate-limiting conversion of single-stranded to double-stranded vector genomes during transduction (Fisher et al., J Virol 70: 520-532, 1996). Preliminary experiments showed that the rAAV-GPE-G6PT vector was also more efficacious than the rAAV-miGT-G6PT vector. Accordingly, the dosages of the two vectors administrated to the G6pt−/− mice were adjusted in this study to yield comparable levels of restoration of hepatic G6PT activity.

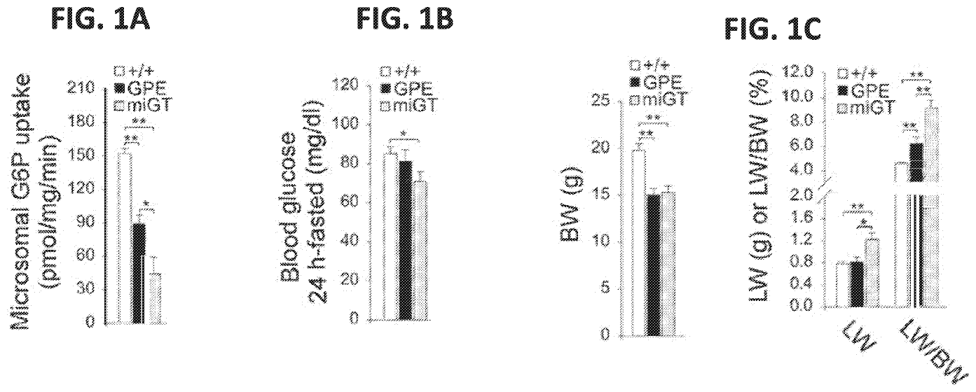

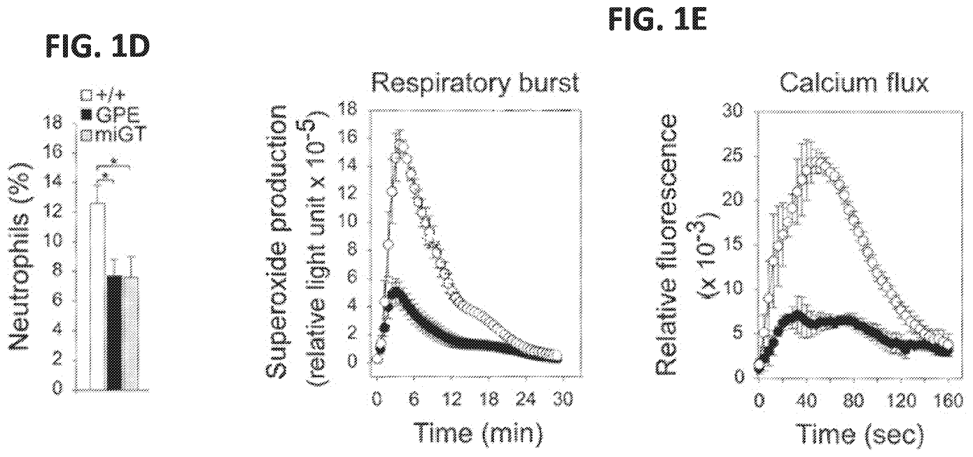

Since GSD-Ib mice die young, early therapeutic intervention is required. However, because of the vector dilution that occurs during the rapid growth of transduced neonatal liver, two serial doses were required to treat the mice effectively. For rAAV-GPE-G6PT, the first (neonatal) dose was 0.7×10¹³ viral particles (vp)/kg followed at 4 weeks with a second dose of 2×10¹³ vp/kg. These mice were called “GPE” mice. For rAAV-miGT-G6PT, both of the doses were two-fold higher than for the GPE mice. These mice were called “miGT” mice. Both vectors delivered the G6PT transgene to the liver of G6pt−/− mice and markedly improved their survival. Hepatic microsomes isolated from 6 week old mice (n=12 per therapy) had G6P uptake activity of 60% (GPE) and 30% (miGT), respectively of wild-type hepatic G6P uptake activity (152±5 units) (FIG. 1A), indicating that the rAAV-GPE-G6PT vector expresses approximately 4-fold more activity than the rAAV-miGT-G6PT vector on a dose (vp/kg) basis. Notably, both GPE and miGT mice could sustain 24 hours of fasting (FIG. 1B). While the 24-hour fasted blood glucose levels of GPE were consistently lower than those of wild-type mice, they were not statistically different. Similarly, the 24-hour fasted blood glucose levels of miGT mice were also lower but still within the normal range (FIG. 1B). Both GPE and miGT mice were significantly leaner than their wild-type control littermates (FIG. 1C). While the liver weights (LW) of GPE mice were similar to that of wild-type mice, the liver weights of miGT mice were significantly higher (FIG. 1C). Because the rAAV-treated mice were leaner, the ratios of LW to body weight (LW:BW) in both mouse groups were higher than that of wild-type littermates (FIG. 1C). GSD-Ib is also characterized by neutropenia and neutrophil dysfunction (Chou et al., Curr Mol Med 2:121-143, 2002; Chou et al., Nat Rev Endocrinol 6: 676-688, 2010). It was previously shown that rAAV-CBA/CMV-G6PT infusion corrects neutropenia in G6pt−/− mice transiently for 2 weeks (Yiu et al., J Hepatol 51: 909-917, 2009). In this study, the 6-week-old GPE and miGT mice continued manifesting neutropenia (FIG. 1D) and neutrophil dysfunction (FIG. 1E). That finding most likely reflects the different cellular tropisms of the AAV2/8 serotype.

rAAV Infusion Directs Long-Term Hepatic G6PT Expression

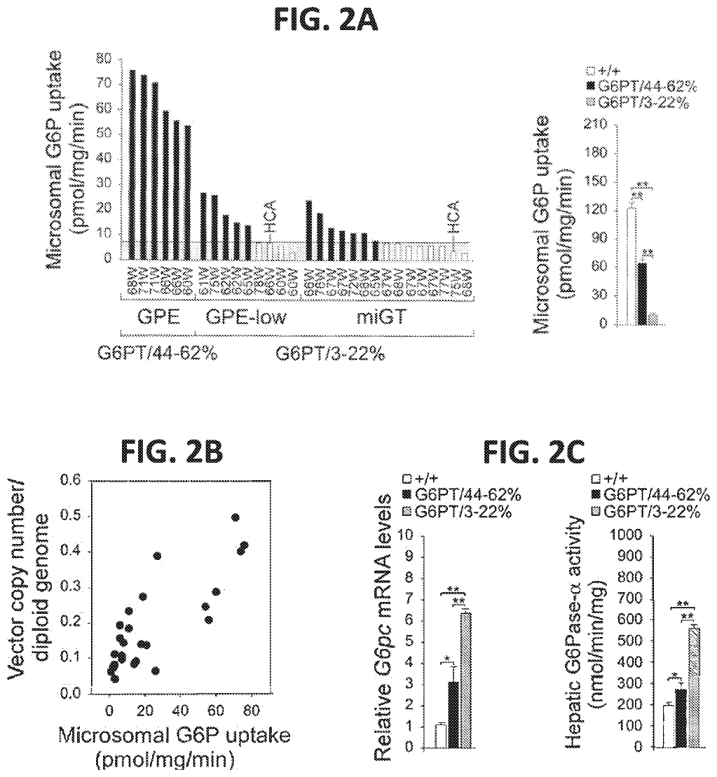

The dosage of the rAAV vectors required to maintain glucose homeostasis and prevent HCA development in G6pt−/− mice was examined over a 78-week study. For the rAAV-GPE-G6PT studies, all neonatal mice (n=15) received 0.7×10¹³ vp/kg followed at 4 weeks by either 2×10¹³ vp/kg (GPE mice, n=6) or 0.7×10¹³ vp/kg (GPE-low mice, n=9). For the rAAV-miGT-G6PT studies, all neonatal mice (n=15) received 1.4×10¹³ vp/kg neonatally, then 4×10¹³ vp/kg at age 4 weeks; these were called “miGT” mice. Hepatic G6PT activity was examined in wild-type and rAAV-treated mice sacrificed after a 24-hour fast. For the 60-78-week-old wild-type mice, the mean hepatic microsomal G6P uptake activity was 123±6 units (or pmol/min/mg) (representing 100% normal hepatic G6PT activity). The GPE mice were titrated to reconstitute 44-62% of wild-type hepatic G6PT activity and were named G6PT/44-62% mice (FIG. 2A). The GPE-low and miGT mice had 3-22% of wild-type hepatic G6PT activity and were named G6PT/3-22% mice (FIG. 2A). There was no HCA in any of the 60-78 week-old wild-type or G6PT/44-62% mice (FIG. 2A). Among the 24 G6PT/3-22% mice, 12 had microsomal G6P uptake activity ≤7 units (or ≤5.7% of normal hepatic G6PT activity). One GPE-low and one miGT mouse with 5.7% and 3.2% of normal hepatic G6P uptake activity, respectively, in the non-tumor liver tissues developed HCA (FIG. 2A). This suggests that 5.7% of normal hepatic G6PT activity is on the threshold of HCA formation in GSD-Ib. The increases in hepatic G6P uptake activity appeared to correlate with the increases in hepatic vector genome copy number (FIG. 2B). In summary, the rAAV-treated G6pt−/− mice with <6% of normal hepatic G6PT activity restored are at risk of developing HCA.

During fasting, blood glucose homeostasis is maintained by hydrolysis of G6P to glucose by the G6PT/G6Pase-α complex in the terminal step of gluconeogenesis and glycogenolysis in the liver (Chou et al., Curr Mol Med 2: 121-143, 2002; Chou et al., Nat Rev Endocrinol 6: 676-688, 2010). It was shown that levels of hepatic G6pc mRNA were increased in all rAAV-treated G6pt−/− mice relative to wild-type mice (FIG. 2C). In parallel, levels of hepatic G6Pase-α enzymatic activity in all rAAV-treated mice were increased 1.4-fold to 2.7-fold over that of wild-type controls (FIG. 2C). The G6PT-mediated hepatic microsomal G6P uptake activity is the rate-limiting step in endogenous glucose production (Arion et al., J Biol Chem 251: 6784-690, 1976) but it is co-dependent on G6Pase-α activity (Lei et al., Nat Genet 13: 203-209, 1996). Previously we have shown that hepatic microsomes prepared from GSD-Ia mice which lack G6Pase-α but express wild-type G6PT, exhibit markedly lower G6P uptake activity compared to wild-type hepatic microsomes (Lei et al., Nat Genet 13: 203-209, 1996). That phenotype can be reversed if G6Pase-α activity is restored via gene transfer (Zingone et al., J Biol Chem 275: 828-832, 2000). In rAAV-treated G6pt−/− mice, the increase in hepatic G6Pase-α activity was inversely correlated to hepatic microsomal G6P uptake activity (compare FIGS. 2A and 2C).

rAAV Infusion Corrects Metabolic Abnormalities in GSD-Ib

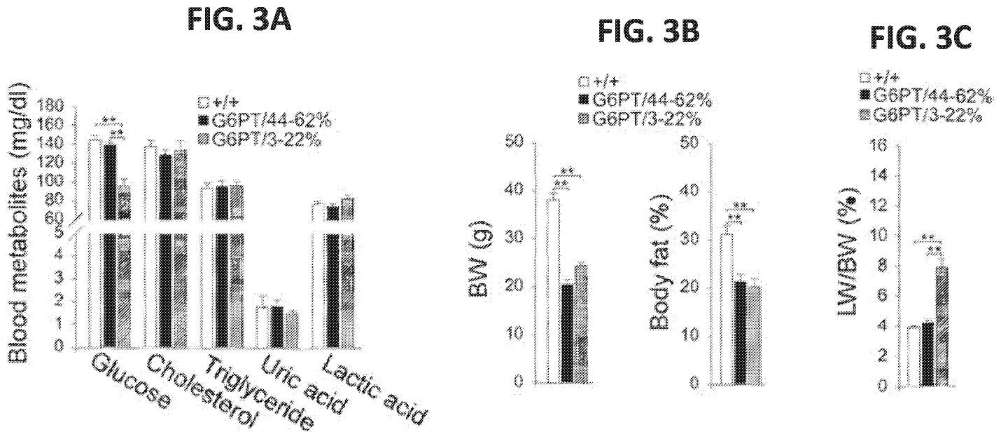

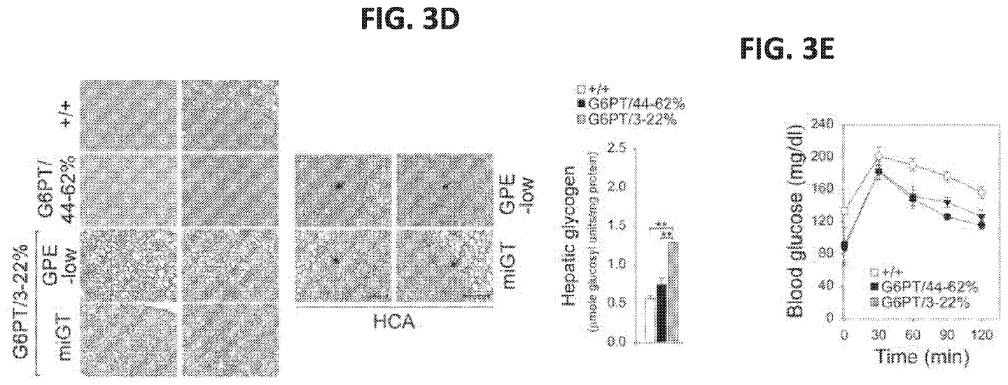

GSD-Ib is characterized by hypoglycemia, hyperlipidemia, hyperuricemia, and lactic acidemia (Chou et al., Curr Mol Med 2: 121-143, 2002; Chou et al., Nat Rev Endocrinol 6: 676-688, 2010). None of the 60-78 week-old rAAV-treated G6pt−/− mice suffered from hypoglycemic seizures. The basal blood glucose levels of G6PT/44-62% and wild-type mice were indistinguishable (FIG. 3A). Despite the ability of the G6PT/3-22% mice to maintain normoglycemia, their basal blood glucose levels were significantly lower than wild-type mice (FIG. 3A). Gene therapy normalized serum cholesterol, triglyceride, uric acid, and lactic acid profiles in all treated mice (FIG. 3A). The average BW and body fat (FIG. 3B) values of treated G6pt−/− mice were significantly lower than those of their age-matched control mice, suggesting the treated mice were protected against age-related obesity. GSD-Ib is also characterized by hepatomegaly (Chou et al., Curr Mol Med 2: 121-143, 2002; Chou et al., Nat Rev Endocrinol 6: 676-688, 2010). The liver to body weight ratios were similar between G6PT/44-62% and wild-type mice, although G6PT/3-22% mice continued manifesting hepatomegaly (FIG. 3C).

Aside from hepatomegaly and instances of HCA, the hepatic tissue histology was unremarkable, even for the non-tumor regions of the two HCA-bearing mice (FIG. 3D). One HCA nodule of 1 cm in diameter was identified in a GPE-low mouse expressing 5.7% of normal hepatic G6PT activity, and 4 HCA nodules of 1, 0.7, 0.3, and 0.3 cm in diameter were identified in a miGT mouse expressing 3.2% of normal hepatic G6PT activity. The HCAs were well circumscribed with increased glycogen storage in both HCA and non-HCA tissues (FIG. 3D). While hepatic glycogen contents of G6PT/44-62% and wild-type mice were statistically similar, the G6PT/3-22% mice exhibited marked increases in glycogen storage (FIG. 3D). The blood glucose tolerance profiles of all treated mice were indistinguishable from those of wild-type littermates (FIG. 3E).

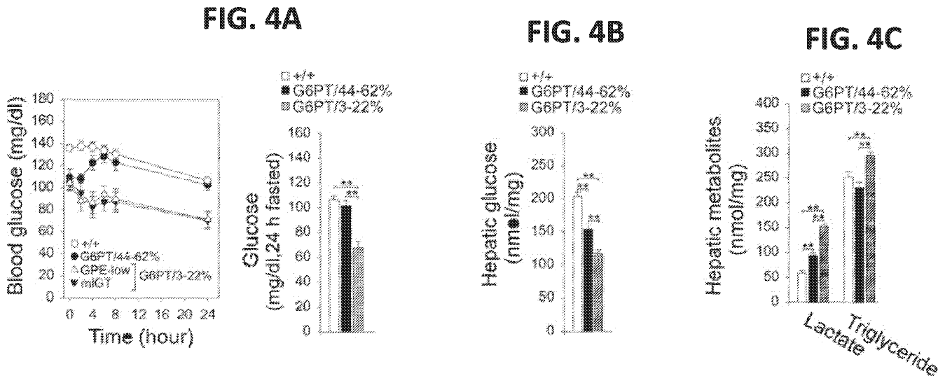

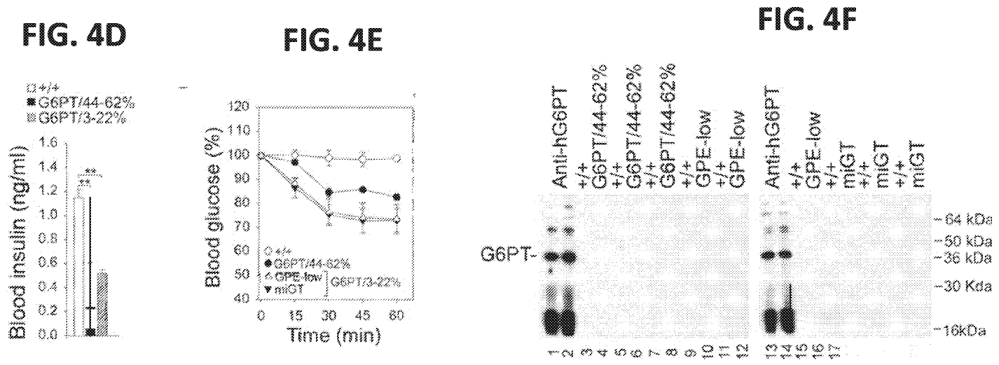

The fasting blood glucose profiles of G6PT/44-62% and wild-type mice were indistinguishable (FIG. 4A). The fasting blood glucose profiles of GPE-low and miGT mice paralleled those of the control mice but blood glucose levels were consistently lower (FIG. 4A). In summary, G6pt−/− mice expressing more than 3% of normal hepatic G6PT activity no longer suffered from the fasting hypoglycemia characteristic of GSD-Ib.

Biochemical Phenotype of the rAAV-Treated G6pt−/− Mice

The G6pt−/− mice, lacking a functional G6PT, are incapable of producing endogenous glucose via the G6PT/G6Pase-α complex. All of the rAAV-treated G6pt−/− mice could tolerate a long fast. Indeed, after 24 hours of fasting, hepatic free glucose levels in G6PT/44-62% and G6PT/3-22% mice were 76%, and 58%, respectively, of wild-type hepatic glucose levels (204±6 nmole/mg) (FIG. 4B). Furthermore, hepatic lactate levels were significantly increased in all rAAV-treated mice but were more pronounced in the G6PT/3-22% mice. While hepatic triglyceride contents were similar between G6PT/44-62% and wild-type mice, hepatic triglyceride levels in G6PT/3-22% mice were significantly increased compared to the controls (FIG. 4C).

Fasting blood insulin levels in the 60-78 week-old wild-type mice were 1.15±0.07 ng/ml (FIG. 4D). Blood insulin levels were significantly lower in all rAAV-treated G6pt−/− mice (FIG. 4D), which were closer to the levels in 10-20 week-old young adult mice than those in the old wild-type mice (Flatt and Bailey, Horm Metab Res 13, 556-560, 1981). The rAAV-treated G6pt−/− mice exhibit increased insulin sensitivity and a reduced insulin dose of 0.25 IU/kg was chosen to monitor blood insulin tolerance profiles. Following an intraperitoneal insulin injection, blood glucose levels in the old wild-type failed to decrease (FIG. 4E). reflecting age-related decrease in insulin sensitivity (Barzilai et al., Diabetes, 61, 1315-1322, 2012). While all treated mice exhibited increased insulin sensitivity as compared to wild-type mice, the increase in insulin sensitivity was more pronounced in the G6PT/3-22% mice (FIG. 4E).

To determine whether a humoral response directed against human G6PT is generated in the infused mice, Western blot analysis was performed using the sera (1:50 dilution) obtained from the 60-78-week-old wild-type and rAAV-treated G6pt−/− mice. A polyclonal anti-human G6PT antibody (Chen et al., Hum Mol Genet 11: 3199-3207, 2002) that also recognizes murine G6PT was used as a positive control (lane 1, 2, 13, 14). No antibodies against G6PT were detected in the sera of the control and rAAV-treated G6pt−/− mice (FIG. 4F).

### Activation of Hepatic ChREBP Signaling

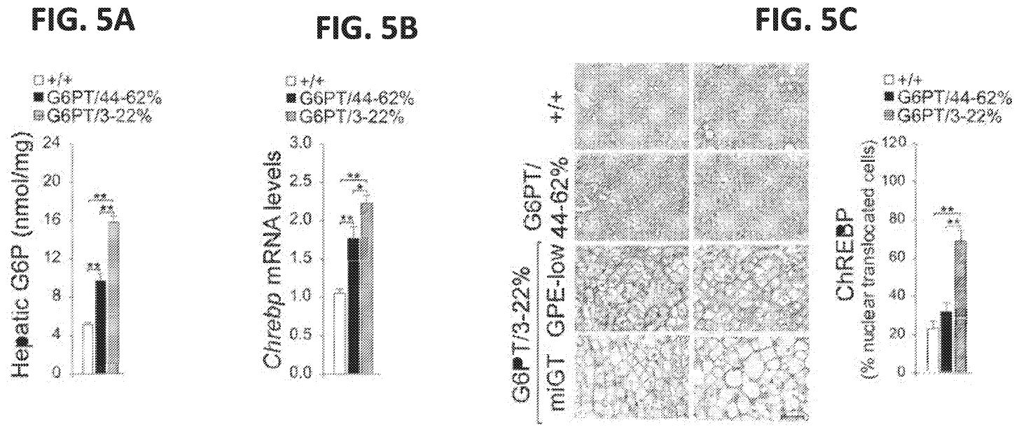

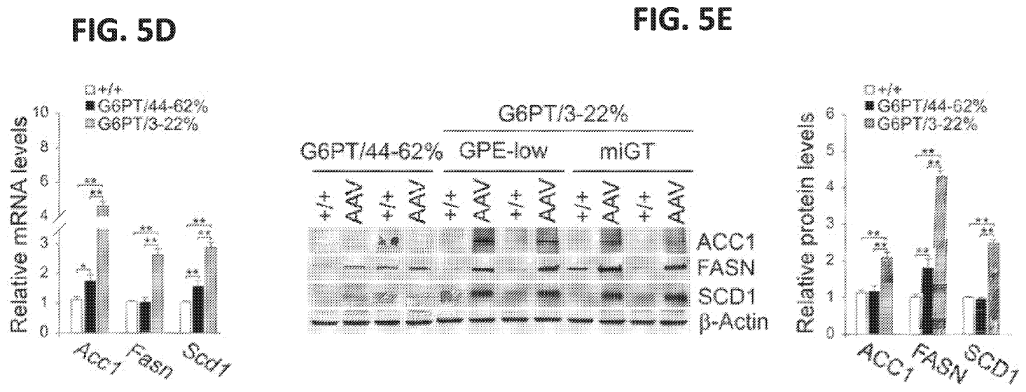

Studies have shown that mice over-expressing hepatic carbohydrate response element binding protein (ChREBP) exhibit improved glucose tolerance compared to controls (Benhamed et al., J Clin Invest 122, 2176-2194, 2012). It has been shown that activation of ChREBP signaling is one pathway that protects the rAAV-treated GSD-Ia mice from developing age-related insulin resistance (Kim et al., Hum Mol Genet 24: 5115-5125, 2015). ChREBP signaling can be activated by G6P, which promotes ChREBP nuclear translocation (Filhoulaud et al., Trends Endocrinol Metab 24, 257-268, 2013). In this study of rAAV-treated G6pt−/− mice, hepatic levels of G6P in G6PT/44-62% and G6PT/3-22% mice were 1.9- and 3.1-fold higher, respectively, than the control mice (FIG. 5A). This was accompanied by increased hepatic Chrebp transcripts in all rAAV-treated G6pt−/− mice (FIG. 5B). Compared to wild-type mice, hepatic nuclear ChREBP protein contents were markedly increased in G6PT/3-22% mice but the increase in hepatic nuclear ChREBP protein contents was not statistically significant in G6PT/44-62% mice (FIG. 5C). Consistently, levels of mRNA and protein of ChREBP-regulated hepatic genes (Benhamed et al., J Clin Invest 122, 2176-2194, 2012; Filhoulaud et al., Trends Endocrinol Metab 24, 257-268, 2013), acetyl-CoA carboxylase isoform-1 (ACC1), fatty acid synthase (FASN), and stearoyl-CoA desaturase 1 (SCD1) were markedly increased in the G6PT/3-22% mice but only moderately and inconsistently increased in the G6PT/44-62% mice (FIGS. 5D and 5E).

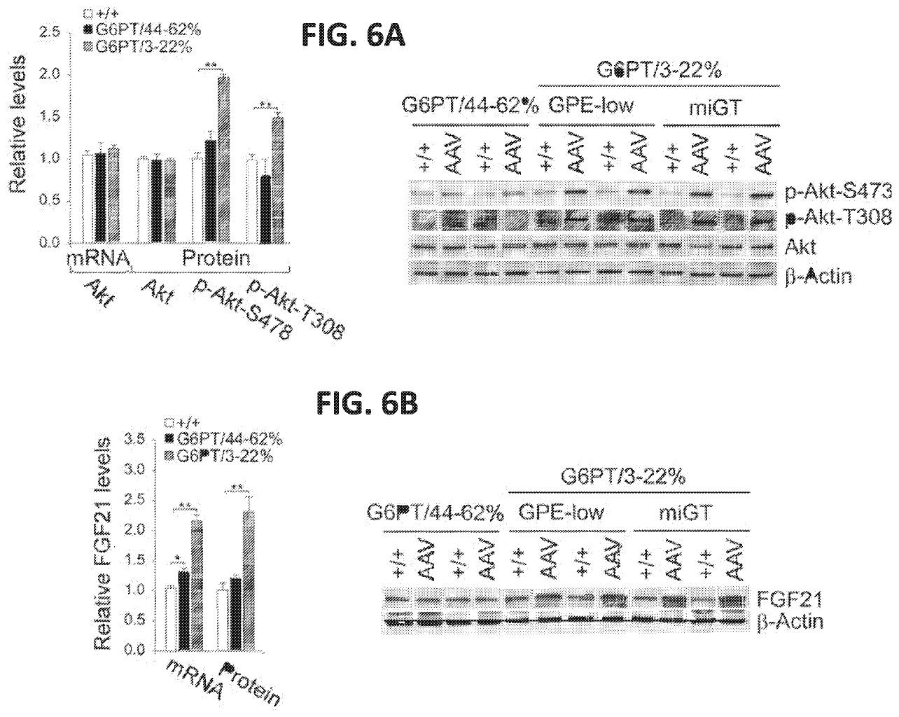

Studies have shown that mice overexpressing hepatic ChREBP along with increased SCD1 exhibit improved insulin signaling that correlates with phosphorylation and activation of protein kinase B/Akt (Benhamed et al., J Clin Invest 122, 2176-2194, 2012). Hepatic Akt mRNA and total Akt protein were similar between wild-type and rAAV-treated G6pt−/− mice (FIG. 6A). In parallel with the increase in hepatic levels of nuclear translocation of ChREBP protein, hepatic levels of the active, phosphorylated forms of Akt (Danielpour and Song, Cytokine Growth Factor Rev 17, 59-74, 2006), p-Akt-S473 and p-Akt-T308, were statistically similar for the wild-type and G6PT/44-62% mice. However, for the G6PT/3-22% mice, while the Akt protein levels remained wild-type, p-Akt-S473 and p-Akt-T308, were 2.1 and 1.5-fold higher (FIG. 6A).

FGF21 is a major regulator of energy homeostasis and insulin sensitivity (Fisher and Maratos-Flier, Annu Rev Physiol 78, 223-241, 2016) and is a target of ChREBP (lizuka et al., FEBS Lett 583, 2882-2886, 2009). The administration of FGF21 reverses hepatic steatosis, counteracts obesity, and alleviates insulin resistance in both rodents and nonhuman primates (Fisher and Maratos-Flier, Annu Rev Physiol 78, 223-241, 2016). Again, consistent with the increase in hepatic levels of nuclear translocation of ChREBP protein, hepatic levels of FGF21 transcript and protein were markedly higher only in G6PT/3-22% mice, compared to the controls (FIG. 6B).

### Therapeutic Applications

Previous gene therapy studies have shown that a G6PT-expressing rAAV2/8 vector directed by the CBA promoter/CMV enhancer delivered the transgene to the liver and achieved metabolic correction in murine GSD-Ib (Yiu et al., J Hepatol 51: 909-917, 2009). While that study showed promise. hepatic G6PT activities restored in the 52-72-week-old G6pt−/− mice were low, averaging approximately 3% of normal hepatic G6PT activity, and 2 of the 5 transduced mice developed multiple HCAs with one undergoing malignant transformation (Yiu et al., J Hepatol 51: 909-917, 2009). Previous studies in hepatic disease have also shown that the use of tissue-specific promoter/enhancer elements can improve expression efficiency and reduce the level of immune response that reduces long-term transgene expression (Ziegler et al., Mol Ther 15: 492-500, 2007; Franco et al., Mol Ther 12: 876-884, 2005). It has been shown that the gluconeogenic tissue-specific G6PC promoter/enhancer is significantly more effective than CBA/CMA in directing persistent hepatic G6Pase-α expression in murine GSD-Ia and that an inflammatory immune response elicited by the vector containing the CBA/CMA elements reduced hepatic transgene expression (Yiu et al., Mol Ther 18:1076-1084, 2010). In the study disclosed herein, the efficacy of rAAV-GPE-G6PT, a single-stranded rAAV vector directed by the G6PC promoter/enhancer (GPE) (Lee et al., Hepatology 56: 1719-1729, 2012; Kim et al., Hum Mol Genet 24: 5115-5125, 2015; Yiu et al., Mol Ther 18:1076-1084, 2010) and rAAV-miGT-G6PT, a double-stranded rAAV vector directed by the native G6PT promoter/enhancer (miGT) (Hiraiwa and Chou, DNA Cell Biol 20: 447-453, 2001), were compared. While both vectors directed persistent hepatic G6PT expression, the vector using the G6PC promoter/enhancer was approximately 4-fold more efficient in transgene expression. on a dose basis, than the vector using the native G6PT promoter/enhancer. It was also shown that the rAAV-treated G6pt−/− mice expressing 3-62% of normal hepatic G6PT activity, grew normally for up to 78 weeks, displayed a normalized metabolic phenotype, had no detectable anti-G6PT antibodies, and were protected against age-related obesity and insulin resistance. Significantly, the studies disclosed herein showed that G6pt−/− mice with <6% of normal hepatic G6PT activity restored were at risk of developing hepatic tumors, establishing the threshold of hepatic G6PT activity required to prevent tumor formation was established.

In contrast to GSD-Ib patients (Chou et al., Curr Mot Med 2: 121-143, 2002; Chou et al., Nat Rev Endocrinol 6: 676-688, 2010) and mice (Chen et al., Hum Mol Genet 12: 2547-2558, 2003), which cannot tolerate a short fast, the mice expressing 3-62% of normal hepatic G6PT activity could sustain 24 hours of fasting. The hydrolysis of cytoplasmic G6P depends upon the functional co-dependence of G6PT and G6Pase-α in the G6PT/G6Pase-α complex (Chou et al., Curr Mol Med 2: 121-143, 2002). In gene therapy studies of murine GSD-Ia lacking G6Pase-α, it has been shown that when 3-63% of normal hepatic G6Pase-α activity was reconstituted, the levels of hepatic G6PT mRNA became elevated 2.2-fold over wild-type (Lee et al., Hepatology 56:1719-1729, 2012). In line with the functional co-dependence of G6PT and G6Pase-α in the G6PT/G6Pase-ca complex, the present studies demonstrated there was a 1.4- to 2.8-fold increase in G6Pase-α expression when G6PT activity was reconstituted to 44-62% and 3-22%. respectively, of normal hepatic activity. The treated GSD-Ib mice produced hepatic endogenous glucose averaging 58 to 76% of control littermates, enabling them to maintain glucose homeostasis during prolonged fasts. Therefore, there appears to be a functional feedback mechanism in which the expression levels of G6Pase-α and G6PT are regulated such that a decrease in one is offset by an increase in the other. This partially compensates for the overall decrease in the G6PT/G6Pase-α complex that occurs in type I GSDs. This extends the understanding of the nature of functional co-dependence of the two components of the G6PT/G6Pase-α complex that maintains interprandial blood glucose homeostasis.

The abnormal metabolic liver phenotype of GSD-Ib is characterized by fasting hypoglycemia, hepatomegaly, hyperlipidemia, hyperuricemia, and lactic acidemia (Chou et al., Curr Mol Med 2: 121-143, 2002; Chou et al., Nat Rev Endocrinol 6: 676-688, 2010). The G6PT/3-22% mice exhibited a normalized metabolic liver phenotype but continued exhibiting hepatomegaly. They also had increased hepatic glycogen and triglyceride contents along with reduced basal and 24-hour fasted blood glucose levels. On the other hand, the G6PT/44-62% mice exhibited a metabolic liver phenotype indistinguishable from that of the wild-type mice, including normal levels of blood glucose and metabolites, normal levels of hepatic glycogen and triglyceride, normal LW/BW. and normal glucose tolerance and fasting glucose tolerance profiles. However, unlike wild-type mice that gain fat and lose insulin sensitivity with age, all treated mice were protected against age-related obesity and insulin resistance, although GSD-Ib mice with 3-22% reconstituted hepatic G6PT activity were more insulin sensitive than the mice with 44-62% of reconstituted hepatic G6PT activity.

Studies have shown that mice overexpressing hepatic ChREBP exhibit improved glucose and lipid metabolism resulting from Akt activation and an increase in the expression of SCD1, which converts saturated fatty acids into the beneficial mono-unsaturated fatty acids (Benhamed et al., J Clin Invest 122, 2176-2194, 2012; Flowers and Ntambi, Curr Opin Lipidol 19:248-256, 2008). Moreover, FGF21, which improves insulin sensitivity, ameliorates hepatic steatosis and enhances energy expenditure (Fisher and Maratos-Flier, Annu Rev Physiol 78, 223-241, 2016), is a target of ChREBP (lizuka et al., FEBS Lett 583, 2882-2886, 2009). The studies disclosed herein demonstrated that hepatic ChREBP signaling is activated in the 60-78-week-old G6PT/3-22% mice, evident by increased nuclear translocation of ChREBP proteins, along with increased levels of FGF21. SCD1, the active p-Akt-S473 and p-Akt-T308, providing one underlying mechanism for the improved metabolic phenotype of the G6PT/3-22% mice. GSD-Ib is an autosomal recessive disorder. It is therefore not surprising that the G6PT/44-62% mice displayed a metabolic liver phenotype indistinguishable from that of wild-type mice. Indeed, ChREBP signaling in G6PT/44-62% and wild-type mice appeared to be similar. Supporting this, the components of the ChREBP signaling pathways, including nuclear translocated ChREBP proteins, activated forms of Akt, and levels of SCD1 and FGP21, were statistically similar between G6PT/44-62% and wild-type mice. This may explain the reduced insulin sensitivity of these mice, compared to G6PT/3-22% mice expressing lower levels of normal hepatic G6PT activity. The fact that the G6PT/3-22% mice exhibited a more improved metabolic phenotype than the G6PT/44-62% mice suggests that semi-optimal levels of hepatic G6PT activity might be beneficial. This reflects a similar observation seen in the GSD-Ia mice (Antinozzi et al., Annu Rev Nuir 19: 511-544, 1999; Clore et al., Diabetes 49: 969-974, 2000). This reflects a similar observation seen in the GSD-Ia mice (Kim et al., Hum Mol Genet 24: 5115-5125, 2015) and perhaps not surprising given the link between increases in hepatic G6Pase-α/G6PT activity and diabetes (Antinozzi et al., Annu Rev Nutr 19: 511-544, 1999; Clore et al., Diabetes 49: 969-974, 2000).

In summary, the studies disclosed herein demonstrated that G6pt−/− mice receiving G6PT gene therapy titrated to express at least 3% of normal hepatic G6PT activity maintain glucose homeostasis and are protected against age-related insulin resistance and obesity. It is further shown that one underlying mechanism responsible for the beneficial metabolic phenotype of the treated mice arises from activation of hepatic ChREBP signaling pathway. Furthermore, hepatocytes harboring less than 6% of normal hepatic G6PT activity are at risk of malignant transformation. These studies indicate that full restoration of normal G6FT activity will not be required to confer significant therapeutic benefits in liver-directed gene therapy for metabolic disease in GSD-Ib.

### Example 3: Analysis of Signaling Pathways in G6PT Transgenic Mice

The rAAV8-mediated G6PT transgene expression primarily targeted the liver and very little transgene expression was observed in the kidney and intestine. Consequently, kidney and intestine of the treated mice remained G6pt-null and incapable of endogenous glucose production. In the absence of endogenous glucose production from the kidney and intestine, the G6PT/3-22% mice produced reduced levels of hepatic glucose averaging 58% of those of control littermates (FIG. 4B), suggesting that the G6PT/3-22% mice mimic animals living under calorie restriction.

AMPK (AMP-activated protein kinase) and SIRT1 (sirtuin 1) are two modulators of calorie restriction that are involved in regulation of energy metabolism (Ruderman et al., Am J Physiol Endocrinol Metab 298: E751-760, 2010). AMPK inhibits interleukin-6-mediated phosphorylation and activation of signal transducer and activator of transcription 3 (STAT3). a cancer-promoting transcription factor (He and Karin, Cell Res 21:159-168, 2011). SIRT1 is a NAD⁺-dependent deacetylase that can be activated at the transcriptional level or in response to an increase in cellular NAD+ levels (Mouchiroud et al., Crit Rev Biochem Mol Biol 48: 397-408, 2013). SIRT1 deacetylates residue K310 on the p65 subunit of nuclear factor iB (NFwB) and represses the activity of NFwB, a transcription factor that regulates inflammation and promotes inflammation-associated cancer (He and Karin, Cell Res 21:159-168, 2011). The signaling by STAT3 and NFwB is highly interconnected (Yu et al., Nat Rev Cancer 9:798-809, 2009). Together they regulate many genes involved in tumor proliferation, survival and invasion. Therefore signaling by AMPK, SIRT1, STAT3 and NFB in G6PT/44-62% and G6PT/3-22% mice was examined.

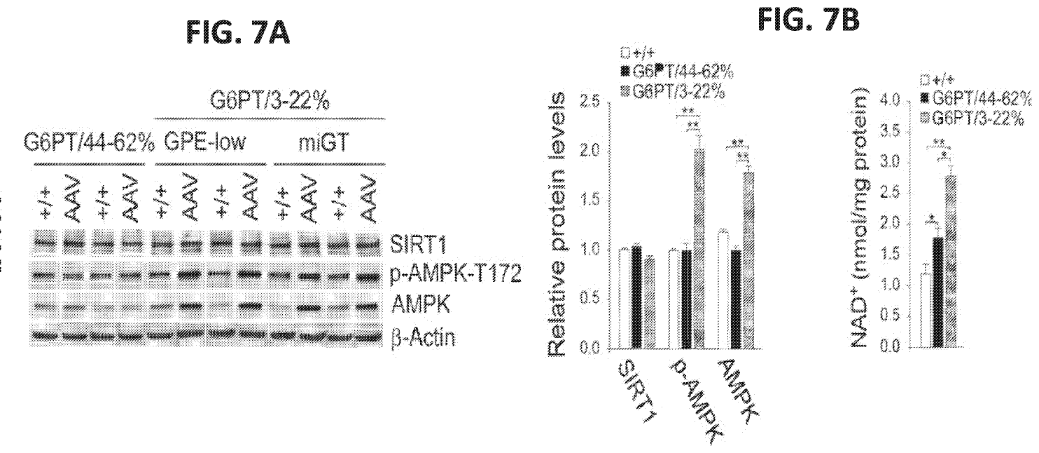

Compared to wild-type mice, hepatic levels of total AMPK and active p-AMPK-T172 were markedly increased in the G6PT/3-22% mice, but not in the G6PT/44-62% mice (FIG. 7A), suggesting activation of AMPK signaling occurred mainly in the G6PT/3-22% mice. While SIRT1 protein levels were similar between wild-type and rAAV-treated mice (FIG. 7A), hepatic NAD⁺ concentrations were markedly increased in the G6PT/3-22% mice and to a lesser extent in the G6PT/44-62% mice (FIG. 7B). This result suggests that hepatic SIRT1 activity is primarily activated in the G6PT/3-22% mice. Taken together, the G6PT/3-22% mice with activated AMPK/SIRT1 signaling displayed a healthy aging phenotype, compared to both wild-type and G6PT/44-62% mice.

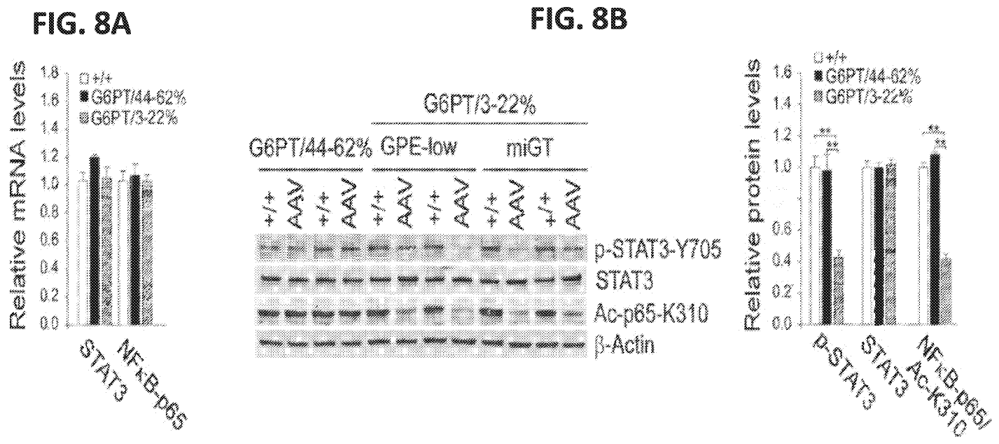

The expression of STAT3 and NFκB were then examined. Both are regulated by the AMPK-SIRT1 signaling pathway. Hepatic levels of STAT3 and NFκB-p65 transcript and the STAT3 protein were not statistically different between rAAV-treated G6pt−/− and wild-type mice (FIGS. 8A-8B). While hepatic levels of the active p-STAT3-Y705 and active ac-NFκB-p65-K310 were similar between G6PT/44-62% and wild-type mice, hepatic levels of p-STAT3-Y705 and ac-NFκB-p65-K310 were significantly reduced in G6PT/3-22% mice compared to both G6PT44-62% and wild-type mice (FIG. 8B). This suggests that the G6PT/3-22% mice also displayed a liver environment with reduced inflammatory and tumorigenic responses.

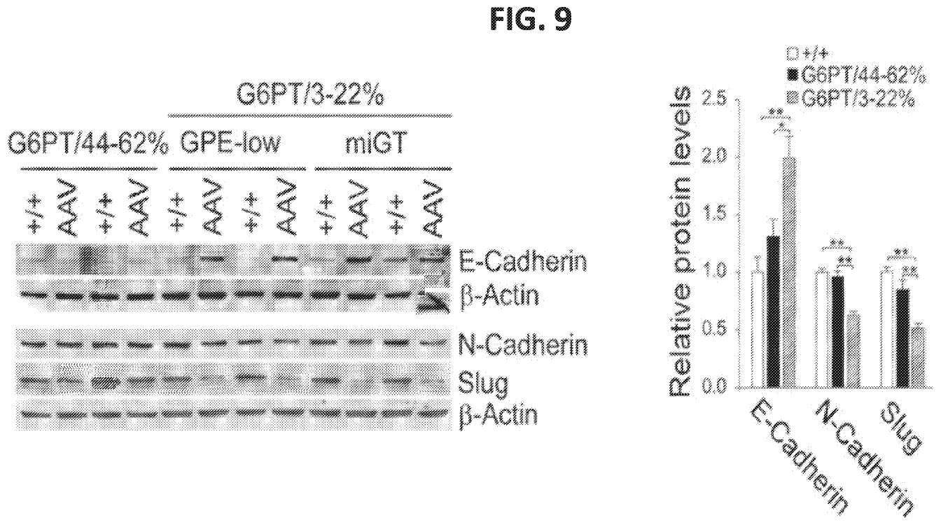

SIRT1 is also a negative regulator of tumor metastasis that increases the expression of E-cadherin, a tumor suppressor, and decreases the expression of mesenchymal markers, including N-cadherin (Chen et al., Mol Cancer 13: 254, 2014). E-Cadherin is a cell-cell adhesion molecule that regulates epithelial-mesenchymal transition (EMlT) and a decrease in E-cadherin expression leads to the initiation of metastasis (Canel et al., J Cell Sci 126(Pt 2):393-401, 2013). Compared to wild-type mice, hepatic protein levels of E-cadherin were markedly increased primarily in G6PT/3-22% mice (FIG. 9). The G6PT/3-22% livers showed decreased protein levels of N-cadherin and the EMT-inducing transcription factor, Slug (FIG. 9). Again, the G6PT/3-22% mice displayed a liver environment with reduced tumorigenic responses.

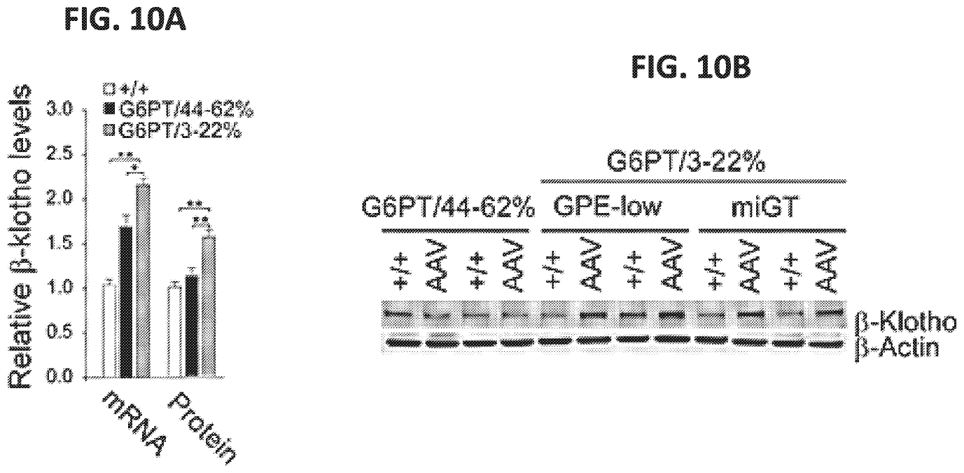

The improved metabolic phenotype of the G6PT/3-22% mice suggests that additional calorie restriction responsive genes may be induced. FGF21, a calorie restriction responsive gene. was shown to be increased in G6PT/3-22% mice (FIG. 6B). Hepatic levels of mRNA and protein for the tumor suppressor β-klotho (Ye et al., PLoS One, 8:e55615, 2013), another calorie restriction responsive gene, were markedly increased in G6PT/3-22% mice, compared to controls (FIGS. 10A-10B).

In summary, the underlying mechanisms responsible for the improved metabolic phenotype of the G6PT/3-22% mice correlate with activation of hepatic AMPK/SIRT1 and FGF21/β-klotho signaling pathways and downregulation of hepatic STAT3/NFκB-mediated inflammatory and tumorigenic signaling pathways. The finding that a moderate reduction of hepatic G6PT activity in mice generates a liver environment with reduced inflammatory and tumorigenic responses provides insight into the biology and pathogenesis of the role of G6PT in hepatic tumorigenesis.

In view of the many possible embodiments to which the principles of the disclosure may be applied, it should be recognized that the illustrated embodiments are only preferred examples of the disclosure and should not be taken as limiting the scope of the disclosure. Rather, the scope of the disclosure is defined by the following claims. We therefore claim all that comes within the scope and spirit of these claims.

1. A recombinant nucleic acid molecule comprising nucleotides 182-4655 of SEQ ID NO: 1 or nucleotides 182-1938 of SEQ ID NO: 2.

2. The recombinant nucleic acid molecule of claim 1, comprising nucleotides 17-5003 of SEQ ID NO: 1 or nucleotides 17-2316 of SEQ ID NO: 2.

3. The recombinant nucleic acid molecule of claim 1, comprising SEQ ID NO: 1 or SEQ ID NO: 2.

4-5. (canceled)

6. A vector comprising the recombinant nucleic acid molecule of claim 1.

7. The vector of claim 6, which is an adeno-associated virus (AAV) vector.

8. The vector of claim 7, wherein the AAV vector is an AAV serotype 8 (AAV8) vector or serotype 9 (AAV9) vector.

9. A recombinant AAV (rAAV) comprising the recombinant nucleic acid molecule of claim 1.

10. The rAAV of claim 9, which is a rAAV8 or rAAV9.

11. The vector of claim 6, which is a lentivirus vector.

12. The vector of claim 11, wherein the lentivirus vector is a human immunodeficiency virus (HIV) vector.

13. A recombinant lentivirus comprising the recombinant nucleic acid molecule of claim 1.

14. The recombinant lentivirus of claim 13, which is a recombinant HIV.

15. A composition comprising the rAAV of claim 9 in a pharmaceutically acceptable carrier.

16. The composition of claim 15 formulated for intravenous administration.

17. A method of treating a subject diagnosed with a glycogen storage disease, comprising selecting a subject with glycogen storage disease type Ib (GSD-Ib) and administering to the subject a therapeutically effective amount of the rAAV of claim 9.

18. The method of claim 17, wherein the rAAV is administered intravenously.

19. The method of claim 17 or claim 18, comprising administering about 1×10¹¹ to about 1×10¹⁴ viral particles (vp)/kg of the rAAV per dose, about 1×10¹² to about 1×10¹⁴ vp/kg of the rAAV per dose, or about 5×10¹² to about 5×10¹³ vp/kg of the rAAV per dose.

20-21. (canceled)

22. The method of claim 17, wherein administering the rAAV comprises administration of a single dose of rAAV.

23. The method of claim 17, wherein administering the rAAV comprises administration of multiple doses of rAAV.

24. A composition comprising the recombinant lentivirus of claim 13 in a pharmaceutically acceptable carrier.

25. The composition of claim 24 formulated for intravenous administration.

26. A method of treating a subject diagnosed with a glycogen storage disease, comprising selecting a subject with glycogen storage disease type Ib (GSD-Ib) and administering to the subject a therapeutically effective amount of the recombinant lentivirus of claim 13.


SEQUENCE LISTING (2 sequences)

SEQ ID NO: 1 - DNA, 7862 bp, Artificial Sequence
  Synthetic construct (pTR-GPE-human G6PT)
  misc_feature (17)..(163): Inverted terminal repeat
  misc_feature (182)..(3045): G6PC promoter/enhancer (GPE)
  misc_feature (3185)..(3321): Intron
  misc_feature (3366)..(4655): G6PT coding sequence
  misc_feature (4868)..(5003): Inverted terminal repeat
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtacccctttgagaatccacggtgtctcgatgcagtcagctttctaacaagctggggcctcacctgttttcccacggataaaaacgtgctggaggaagcagaaaggggctggcaggtggaaagatgaggaccagctcatcgtctcatgactatgaggttgctctgatccagagggtccccctgcctggtggcccaccgccaggaagactcccactgtccctggatgcccagagtgggatgtcaactccatcacttatcaactccttatccataggggtattcttcctgaggcgtctcagaaaacagggccctccccatatgctgaccacataatagaacccctcccaactcagagaccctggctgctagctgccctggcatgacccagacagtggcctttgtatatgtttttagactcaccttgactcacctctgaccatagaaactctcatcccagaggtcactgcaatagttactccacaacagaggcttatctgggtagagggaggctccctacctatggcccagcagccctgacagtgcagatcacatataccccacgccccagcactgcctgccacgcatgggcttactttacacccacccacagtcaccaacacattacctgctctccaaggttaggcgtggcaggagaagtttgcttggaccagcagaaaccatgcagtcaaggacaactggagtcagcatgggctgggtgcgagcccttggtggggtggggaggagactccaggtcatacctcctggaggatgttttaatcatttccagcatggaatgctgtcaacttttgccacagattcattagctctgagtttcttttttctgtccccagctaccccttacatgtcaatatggacttaatgatgggaaattcaggcaagtttttaaacattttattccccctggctcttatcctcaaaaaatgcatgaatttggaggcagtggctcatgcctgtaatcccaatgctttgctaggttgaggcgggaggatcacttgaagccaggaatttgagaccagcctgggccgcatagtgagaccccgtttctacaaaaataaataaataaataataaataatagtgatatgaagcatgattaaatagccctattttttaaaatgcatgagttcgttacctgattcattccctggttcctttcacagtcctccgtgacccaagtgttagggttttggtctctctactatttgtaggctgatatatagtatacacacacacacacacacacatatacacacacacagtgtatcttgagctttcttttgtatatctacacacatatgtataagaaagctcaagatatagaagccctttttcaaaaataactgaaagtttcaaactctttaagtctccagttaccattttgctggtattcttatttggaaccatacattcatcatattgttgcacagtaagactatacattcattattttgcttaaacgtatgagttaaaacacttggccaggcatggtggttcacacctgtaatcccagagctttgggaagccaagactggcagatctcttgagctcaggaattcaagaccagcctgggcaacatggaaaaaccccatctctacaaaagatagaaaaattagccaggcatggtggcgtgtgcctgtggtcccagctactcaggaggctgaggtgggaggatcacattagcccaggaggttgaggctgcagtgagccgtgattatgccactgcactccagcctgggagacagagtgagaccctgtttcaaaaaaaagagagagaaaatttaaaaaagaaaacaacaccaagggctgtaactttaaggtcattaaatgaattaatcactgcattcaaaaacgattactttctggccctaagagacatgaggccaataccaggaagggggttgatctcccaaaccagaggcagaccctagactctaatacagttaaggaaagaccagcaagatgatagtccccaatacaatagaagttactatattttatttgttgtttttcttttgttttgttttgttttgttttgttttgttttagagactggggtcttgctcgattgcccaggctgtagtgcagcggtgggacaatagctcactgcagactccaactcctgggctcaagcaatcctcctgcctcagcctcctgaatagctgggactacaagggtacaccatcacacacaccaaaacaattttttaaatttttgtgtagaaacgagggtcttgctttgttgcccaggctggtctccaactcctggcttcaagggatcctcccacctcagcctcccaaattgctgggattacaggtgtgagccaccacaaccagccagaactttactaattttaaaattaagaacttaaaacttgaatagctagagcaccaagatttttctttgtccccaaataagtgcagttgcaggcatagaaaatctgacatctttgcaagaatcatcgtggatgtagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcctgaggtgccaaggaaatgaggtctagagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggccctgcattaccatggcagcccagggctatggctattatcgcactgtgatcttctcagccatgtttgggggctacagcctgtattacttcaatcgcaagaccttctcctttgtcatgccatcattggtggaagagatccctttggacaaggatgatttggggttcatcaccagcagccagtcggcagcttatgctatcagcaagtttgtcagtggggtgctgtctgaccagatgagtgctcgctggctcttctcttctgggctgctcctggttggcctggtcaacatattctttgcctggagctccacagtacctgtctttgctgccctctggttccttaatggcctggcccaggggctgggctggcccccatgtgggaaggtcctgcggaagtggtttgagccatctcagtttggcacttggtgggccatcctgtcaaccagcatgaacctggctggagggctgggccctatcctggcaaccatccttgcccagagctacagctggcgcagcacgctggccctatctggggcactgtgtgtggttgtctccttcctctgtctcctgctcatccacaatgaacctgctgatgttggactccgcaacctggaccccatgccctctgagggcaagaagggctccttgaaggaggagagcaccctgcaggagctgctgctgtccccttacctgtgggtgctctccactggttaccttgtggtgtttggagtaaagacctgctgtactgactggggccagttcttccttatccaggagaaaggacagtcagcccttgtaggtagctcctacatgagtgccctggaagttgggggccttgtaggcagcatcgcagctggctacctgtcagaccgggccatggcaaaggcgggactgtccaactacgggaaccctcgccatggcctgttgctgttcatgatggctggcatgacagtgtccatgtacctcttccgggtaacagtgaccagtgactcccccaagctctggatcctggtattgggagctgtatttggtttctcctcgtatggccccattgccctgtttggagtcatagccaacgagagtgcccctcccaacttgtgtggcacctcccacgccattgtgggactcatggccaatgtgggcggctttctggctgggctgcccttcagcaccattgccaagcactacagttggagcacagccttctgggtggctgaagtgatttgtgcggccagcacggctgccttcttcctcctacgaaacatccgcaccaagatgggccgagtgtccaagaaggctgagtgagcggccgcgcatgataagatacattgatgaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgaccatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatcggcaaacaaaccaccgctggtagcggtggtttttttgtttgcaagcagcagattacgcgcagaaaaaaaggatctcaagagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 2 - DNA, 5157 bp, Artificial Sequence
  Synthetic construct (pTR-miGT-human G6PT)
  misc_feature (17)..(163): Inverted terminal repeat
  misc_feature (182)..(792): miGT
  misc_feature (924)..(1560): Intron
  misc_feature (1105)..(1938): G6PT coding sequence
  misc_feature (2171)..(2316): Inverted terminal repeat
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccctagaaggcttagaagtggggtagccggggtatgtgtctgactttgtgttttggtgcggtggtggtgagtgggtgtagaaactaatagtgccaagtgcagtgcctgtcaactagtaagttaataaaccttccgtgaatgaatgagaaggttctgtgactgagggagcagggctagagagggcatctctgctcatcccacccctgcctcccctgggctcaggcagacaaaagtaattaggttgaacctttaatcaaagctggtttcacaggcaagcagacatgatagaggaggtaaaggcagaaatcctggggacacagggtgctggcctggctcacaggcatgcctccttccgggacctcctccaccccctacagtttggcgctcagtaatctcttgttttcttgtctccctcaggacactgggtccccttggagcctccccaggcttaatgattgtccagaaggcggctataaagggagcctgggaggctgggtggaggagggagcagaaaaaacccaactcagcagatctgggaactgtgagagcggcaagcaggaactgtggtcagaggctgtgcgtcttggctggtagggcctgctcttttctaccaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggccctgcattaccatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtggttgggattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacttcttgtccttctgcaagagtgctgtagtgcccctggcatccgtcagtgtcatcccctactgcctcgcccaggtcctgggccagccgcacaagaagtcgttgtaagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgaccatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgcgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctagggaagggcgatcggtgcgggcctcttcgctattacgccaggctgca